(19) United States
(12) Patent Application Publication
(10) Pub. No.: US 2024/0390735 A1
(43) Pub. Date: Nov. 28, 2024

(54) FACILITATION OF AVATAR GENERATION BY A METRIC DEVICE

(71) Applicant: Braxton K. Davis, Atlanta, GA (US)

(72) Inventor: Braxton K. Davis, Atlanta, GA (US)

(21) Appl. No.: 18/797,896

(22) Filed: Aug. 8, 2024

Related U.S. Application Data
(63) Continuation-in-part of application No. 17/141,140, filed on Jan. 4, 2021, now abandoned.
Application No. 17/141,140 is a continuation-in-part of application No. 17/140,118, filed on Jan. 3, 2021, now abandoned.
Application No. 17/140,118 is a continuation of application No. 16/051,450, filed on Jul. 31, 2018, now Pat. No. 10,918,909.
Application No. 16/051,450 is a continuation of application No. 15/784,175, filed on Oct. 15, 2017, now Pat. No. 10,065,076.
Application No. 15/784,175 is a continuation-in-part of application No. 15/340,966, filed on Nov. 1, 2016, now Pat. No. 10,625,120.

(51) Int. Cl. A63B 24/00; A63B 21/062; A63B 21/072; A63B 21/075; A63B 71/06; G08C 17/02

(52) U.S. Cl. CPC A63B 24/0075; A63B 21/0628; A63B 21/063; A63B 21/0724; A63B 21/0726; A63B 21/075; A63B 24/0062; A63B 24/0087; A63B 71/0622; G08C 17/02; A63B 2024/0068; A63B 2071/063; A63B 2209/08; A63B 2220/17; A63B 2220/40; A63B 2220/805; A63B 2225/50; A63B 2230/06; A63B 2230/75; G08C 2200/00

(57) ABSTRACT

A more efficient exercise system can be facilitated by interactive components. The interactive components can communicate with mobile devices, users, server devices, weight lifting machines, etc. The interactive components can also generate exercise data and provide prompts to a user to facilitate a more efficient workout in addition to facilitating generation of an avatar representative of a user. Additionally, third-parties can receive user workout data and manipulate, or provide guidance to the user to manipulate, exercise equipment remotely to assist a user in attaining his or her goals.

### CROSS-REFERENCE TO RELATED APPLICATIONS

This patent application is a continuation-in-part, and claims the benefit of priority to each of U.S. provisional Patent Application No. 62/957,284, filed on Jan. 5, 2020, and entitled “FACILITATION OF DATA ACCURACY FOR A METRIC DEVICE”, U.S. patent application Ser. No. 17/140,118, filed on Jan. 3, 2021, and entitled “FACILITATION OF INTERACTIVE EXERCISE SYSTEM”, U.S. patent application Ser. No. 17/141,140, filed on Jan. 4, 2021, and entitled “FACILITATION OF DATA ACCURACY FOR A METRIC DEVICE,” (which is a continuation-in-part of patent application Ser. No. 17/140,118 (now abandoned), U.S. patent application Ser. No. 16/051,450 (now U.S. Pat. No. 10,918,909), filed on Jul. 31, 2018, and entitled “FACILITATION OF INTERACTIVE EXERCISE SYSTEM”, U.S. patent application Ser. No. 15/784,175 (now U.S. Pat. No. 10,065,076), filed on Oct. 15, 2017, and entitled “FACILITATION OF INTERACTIVE EXERCISE SYSTEM” (which is a continuation-in-part of patent application Ser. No. 15/340,966 (now U.S. Pat. No. 10,625,120)), filed on Nov. 1, 2016, and entitled “FACILITATION OF INTERACTIVE EXERCISE SYSTEM”. The entirety of the foregoing applications are hereby incorporated by reference herein.

### TECHNICAL FIELD

This disclosure relates generally to wireless communication for exercise-based metrics. More specifically, this disclosure relates to facilitating adjustment of exercise equipment and fitness regimens based on exercise metrics transmitted via an interactive exercise system.

### BACKGROUND

Weightlifting and personal fitness is a growing industry, and there is an increased effort for individuals to be more health conscious. However, at times, it can be difficult to maintain a consistent workout regimen. Although the daily variables of life can negatively alter a person's workout regimen, inconsistent tracking of progress can also negatively affect a person's workout regimen. Consistent tracking of exercise progress can positively influence a person's health, recovery, physical therapy, insurance premiums, and confidence.

The above-described background relating to facilitation of an interactive exercise system is merely intended to provide a contextual overview of some current issues, and is not intended to be exhaustive. Other contextual information may become further apparent upon review of the following detailed description.

### BRIEF DESCRIPTION OF THE DRAWINGS

Non-limiting and non-exhaustive embodiments of the subject disclosure are described with reference to the following figures, wherein like reference numerals refer to like parts throughout the various views unless otherwise specified.

FIG. 1 illustrates an example metric device according to one or more embodiments.

FIG. 2 illustrates an example exercise equipment comprising a metric device in communication with a mobile device according to one or more embodiments.

FIG. 3 illustrates an example exercise equipment comprising a metric device in communication with a mobile device according to one or more embodiments.

FIG. 4 illustrates an example first exercise equipment in communication with a second exercise equipment comprising one or more metric devices in communication with a mobile device according to one or more embodiments.

FIG. 5 illustrates an example exercise equipment comprising a metric device in communication with a mobile device according to one or more embodiments.

FIG. 6 illustrates an example exercise equipment comprising a metric device in communication with a mobile device according to one or more embodiments.

FIG. 7 illustrates an example exercise equipment comprising a metric device in communication with a mobile device and in communication with a wireless network according to one or more embodiments.

FIG. 8A illustrates a side view of an example weight stack and a metric device according to one or more embodiments.

FIG. 8B illustrates a front view of an example weight stack and a metric device according to one or more embodiments.

FIG. 9A illustrates an example perspective view of a barbell, weight plate, and rack according to one or more embodiments.

FIG. 9B illustrates an example perspective view of a barbell, weight plate, rack, and barbell sleeve device according to one or more embodiments.

FIG. 9C illustrates an example perspective view of a barbell, weight plate, rack, and barbell sleeve device according to one or more embodiments.

FIG. 10 illustrates an example perspective view of an example dumbbell rack comprising dumbbells according to one or more embodiments.

FIG. 11 illustrates is an example perspective view of a plate loaded machine and bumper device according to one or more embodiments.

FIG. 12 illustrates an example flow diagram of a method for determining repetition thresholds according to one or more embodiments.

FIG. 13 illustrates an example flow diagram of a system for determining repetition thresholds according to one or more embodiments.

FIG. 14 illustrates an example flow diagram of a machine-readable medium for determining repetition thresholds according to one or more embodiments.

FIG. 15 illustrates an example block diagram of an example mobile handset operable to engage in a system architecture that facilitates secure wireless communication according to one or more embodiments described herein.

FIG. 16 illustrates an example block diagram of an example computer operable to engage in a system architecture that facilitates secure wireless communication according to one or more embodiments described herein.

### DETAILED DESCRIPTION

In the following description, numerous specific details are set forth to provide a thorough understanding of various embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment,” or “an embodiment,” means that a particular feature, structure, or characteristic described in connection with the embodiment is included in at least one embodiment. Thus, the appearances of the phrase “in one embodiment,” “in one aspect,” or “in an embodiment,” in various places throughout this specification are not necessarily all referring to the same embodiment. Furthermore, the particular features, structures, or characteristics may be combined in any suitable manner in one or more embodiments.

As utilized herein, terms “component,” “system,” “interface,” and the like are intended to refer to a computer-related entity, hardware, software (e.g., in execution), and/or firmware. For example, a component can be a processor, a process running on a processor, an object, an executable, a program, a storage device, and/or a computer. By way of illustration, an application running on a server and the server can be a component. One or more components can reside within a process, and a component can be localized on one computer and/or distributed between two or more computers.

Further, these components can execute from various machine-readable media having various data structures stored thereon. The components can communicate via local and/or remote processes such as in accordance with a signal having one or more data packets (e.g., data from one component interacting with another component in a local system, distributed system, and/or across a network, e.g., the Internet, a local area network, a wide area network, etc. with other systems via the signal).

As another example, a component can be an apparatus with specific functionality provided by mechanical parts operated by electric or electronic circuitry; the electric or electronic circuitry can be operated by a software application or a firmware application executed by one or more processors; the one or more processors can be internal or external to the apparatus and can execute at least a part of the software or firmware application. As yet another example, a component can be an apparatus that provides specific functionality through electronic components without mechanical parts; the electronic components can include one or more processors therein to execute software and/or firmware that confer(s), at least in part, the functionality of the electronic components. In an aspect, a component can emulate an electronic component via a virtual machine, e.g., within a cloud computing system.

The words “exemplary” and/or “demonstrative” are used herein to mean serving as an example, instance, or illustration. For the avoidance of doubt, the subject matter disclosed herein is not limited by such examples. In addition, any aspect or design described herein as “exemplary” and/or “demonstrative” is not necessarily to be construed as preferred or advantageous over other aspects or designs, nor is it meant to preclude equivalent exemplary structures and techniques known to those of ordinary skill in the art. Furthermore, to the extent that the terms “includes,” “has,” “contains,” and other similar words are used in either the detailed description or the claims, such terms are intended to be inclusive-in a manner similar to the term “comprising” as an open transition word-without precluding any additional or other elements.

As used herein, the term “weight” can generally refer to weight that a user is lifting, a plate that can be applied to a barbell, a stack of plates, or anything that causes a resistance experience by a person during the person's exercise routine. It should also be noted that in other scenarios of this disclosure, the term “weight” can refer to a user's body weight, where appropriate.

As used herein, the term “infer” or “inference” refers generally to the process of reasoning about, or inferring states of, the system, environment, user, and/or intent from a set of observations as captured via events and/or data. Captured data and events can include user data, device data, environment data, data from sensors, sensor data, application data, implicit data, explicit data, etc. Inference can be employed to identify a specific context or action, or can generate a probability distribution over states of interest based on a consideration of data and events, for example.

Inference can also refer to techniques employed for composing higher-level events from a set of events and/or data. Such inference results in the construction of new events or actions from a set of observed events and/or stored event data, whether the events are correlated in close temporal proximity, and whether the events and data come from one or several event and data sources. Various classification schemes and/or systems (e.g., support vector machines, neural networks, expert systems, Bayesian belief networks, fuzzy logic, and data fusion engines) can be employed in connection with performing automatic and/or inferred action in connection with the disclosed subject matter.

In addition, the disclosed subject matter can be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, computer-readable carrier, or computer-readable media. For example, computer-readable media can include, but are not limited to, a magnetic storage device, e.g., hard disk; floppy disk; magnetic strip(s); an optical disk (e.g., compact disk (CD), a digital video disc (DVD), a Blu-ray Disc™ (BD)); a smart card; a flash memory device (e.g., card, stick, key drive); and/or a virtual device that emulates a storage device and/or any of the above computer-readable media.

As an overview, various embodiments are described herein to facilitate a seamless handoff of communication between mobile devices, metric devices, exercise equipment, and other network devices.

For simplicity of explanation, the methods (or algorithms) are depicted and described as a series of acts. It is to be understood and appreciated that the various embodiments are not limited by the acts illustrated and/or by the order of acts. For example, acts can occur in various orders and/or concurrently, and with other acts not presented or described herein. Furthermore, not all illustrated acts may be required to implement the methods. In addition, the methods could alternatively be represented as a series of interrelated states via a state diagram or events. Additionally, the methods described hereafter are capable of being stored on an article of manufacture (e.g., a machine-readable storage medium) to facilitate transporting and transferring such methodologies to computers. The term article of manufacture, as used herein, is intended to encompass a computer program accessible from any computer-readable device, carrier, or media, including a non-transitory machine-readable storage medium.

It is noted that although various aspects and embodiments are discussed herein with respect to Universal Mobile Telecommunications System (UMTS) and/or Long Term Evolution (LTE), the disclosed aspects are not limited to a UMTS implementation and/or an LTE implementation. For example, aspects or features of the disclosed embodiments can be exploited in substantially any wireless communication technology. Such wireless communication technologies can include UMTS, Code Division Multiple Access (CDMA), Wi-Fi, Worldwide Interoperability for Microwave Access (WiMAX), General Packet Radio Service (GPRS), Enhanced GPRS, Third Generation Partnership Project (3GPP), LTE, Third Generation Partnership Project 2 (3GPP2) Ultra Mobile Broadband (UMB), High Speed Packet Access (HSPA), Evolved High Speed Packet Access (HSPA+), High-Speed Downlink Packet Access (HSDPA), High-Speed Uplink Packet Access (HSUPA), ZigBee, or another IEEE 802.XX technology. Additionally, substantially all aspects disclosed herein can be exploited in legacy telecommunication technologies.

Described herein are systems, methods, articles of manufacture, and other embodiments or implementations that can facilitate interactive exercise equipment. Facilitating interactive exercise equipment can be implemented in connection with any type of device with a connection to the communications network such as: a smart watch, a laptop, a handheld device, a desktop computer, a television, an Internet enabled television, a mobile phone, a smartphone, a tablet user computer (PC), a digital assistant (PDA), a heads up display (HUD), a virtual reality (VR) headset, an augmented reality (AR) headset, another type of wearable computing device, etc.

As fitness and fitness trends increase, systems, methods, and devices can be employed to assist in the fitness and personal development of individuals. Coordinated tracking and output of fitness data can result in refined fitness programs specific to the an individual and his/her their fitness needs. This disclosure details metric devices and systems for tracking and disseminating fitness metrics associated with individuals and/or a group of individuals. It should be noted that functionality between the metric devices and the system can be interchangeable based on the various scenarios discussed herein.

The metric device can procure and generate data in conjunction with exercise equipment. Furthermore, the metric device can be a part of a system comprising servers, mobile devices, other metric devices, cloud-based storage, etc. The number of times a weight or pulley (e.g., barbell, free weight, exercise machine, etc.) is lifted can be detected and/or calculated and specific outputs can be generated in accordance with the detected and/or calculated data. This can help standardize workout regimens and produce better fitness results. Generally, when an individual works out, he/she either have to remember what he/she did previously or write it down so that he/she can pick up where they left off during their next workout session. Consequently, someone who does a variety of different workouts can lose track of how many repetitions (rep) they performed and at what weight and/or resistance level they performed the repetitions. Additionally, it can be cumbersome for someone to keep track of this data by writing it down or storing it in their mobile device.

The metric device can store and/or generate exercise metrics associated with the exercise equipment including, but not limited to: weight data, resistance data, motion data, repetition data, time data, location data, etc. The metric device can also communicate with any other wireless network device via Bluetooth, iBeacon (or similar technology), cellular, zig-bee, Wi-Fi, and/or any other wireless communications standard. The metric device can also comprise a variety of form factors (e.g., a weight selector pin, a barbell sleeve device, a dumbbell disk, and/or a plate loaded machine bumper). In another embodiment, the metric device can comprise stretch sensors and/or other sensors that have the capability to detect the stretch of a resistance band and thereby communicate data (e.g., resistance, strength, stretch, etc.) to a user's mobile phone. It should be understood that the metric device can be separate and distinct from the exercise equipment or the metric device can be built into the exercise equipment. Although the metric device can be separate and distinct from the exercise equipment, it should also be understood that in certain embodiments the metric device can be attached to the exercise equipment. It should also be noted that the metric device can wirelessly communicate with the exercise equipment and/or a mobile device when it is separate and distinct from the exercise equipment. The metric device can generate exercise metrics as a person is exercising. For instance, weight data and repetition data of a user who is bench pressing can be generated to indicate that the user is bench pressing one hundred thirty-five pounds, ten times, within twenty seconds.

Additionally, the metric device can receive inputs (e.g., caloric consumption, sleep time, age, weight, gender, height, ethnic background, nutrition, hydration, etc.) from a mobile device and/or a server device to facilitate generation of biometric data including, but not limited to: caloric expenditure, heart rate, pulse etc. The inputs can also be stored within a user profile (e.g., user identity) of a user, on a server device provided in a data center having multiple servers (e.g., a single rack of servers to hundreds or thousands of servers), a laptop computer, a mobile device, and/or a cloud-based system. For example, the user can access and update the user profile from an application on the mobile device. Alternatively, the user profile can be stored locally on a memory of the mobile device. The inputs, in conjunction with exercise metrics generated and/or received by the metric device can be used to generate additional fitness metrics to assist in a user's fitness regimen. For instance, based on an input of the user's weight, resistance, and repetition data generated by the metric device, the metric device can generate an approximate caloric expenditure of the user during the user's workout session. It should be noted that the additional fitness metrics (e.g., date, time, location, duration, efficiency, progress, etc.) can be generated by the metric device and sent to the mobile device and/or the additional fitness metrics can be generated by a server and/or the mobile device after receiving the exercise metrics from the metric device. Consequently, some operations can be performed at the metric device, the mobile device, the server device, and/or a third-party device. For example, the user's heart rate can be calculated via a wearable device such as an Apple Watch®. The heart rate data from the Apple Watch® can then be used in conjunction with the metrics provided from the metric device to calculate a caloric expenditure of the user. The calculation can be performed at the mobile device, the metric device, the server device, and/or the third-party device. For ease of use, the Apple Watch® could also be used to identify which machine or which exercise a person is performing by placing the Apple Watch® near an RFID tag. The RFID data can then be utilized by the system to allocate and/or sync data from the metric device to a specific user, exercise, and/or machine.

In another embodiment, the additional fitness metrics can be generated at a remote server that can receive exercise metrics from one or more metric devices and/or profile data from the user profile. The additional fitness metrics can provide the user with additional goals and instructions regarding their fitness regimen. For example, if the server device receives user profile data and exercise metric data from one or more metric devices indicating that the user has expended three hundred calories, the server device can generate nutritional and/or nourishment data representative of a meal or liquids that the user should consume to maximize the effect of the workout. This nutritional and/or nourishment data can then be sent to the user's mobile device and/or an application on the mobile device for access by the user.

The metric device can send exercise metrics to a user's mobile device and a remote server for access by another user and/or a group of users. It should be noted that the metric device can send all of the exercise metric data to a server, where the exercise metric data can be pulled down to various mobile devices. A third-party who receives the exercise metric data can be a personal trainer, a physician, a physical therapist, a health insurance company, or the like. Upon accessing the exercise metrics, the third-party can analyze and input goal data to communicate specific goals for a user's current workout session and/or a future workout session. For example, based on exercise metric data, which indicates that the user lifted one hundred thirty-five pounds ten times, the personal trainer can provide a goal, via the system, for the user to lift one hundred forty-five pounds ten times in real-time and/or during a future workout. The system can also generate goal data based on developed algorithms (e.g., AI) and disseminate it to the user's mobile device in real-time and/or for the future. The goal data can be based on a user inputs, third-party inputs (e.g., personal trainer, physical therapist, healthcare company, etc.) an algorithm, etc. Additionally, the system and/or the third-party can prompt the user to perform certain exercises, set or adjust the weight and/or resistance on the exercise equipment, and/or create a workout regimen for the user to follow. It should be noted that the system can comprise one or more metric devices, mobile devices, server devices, etc. all operable to wirelessly communicate with each other. In some embodiments, a personal trainer's or 3^{rd} parties voice can be utilized to provide audible guidance given by the personal trainer or 3^{rd} party themselves. The personal trainer or 3^{rd} parties voice may also be used to overlay the AI such that goals presented by the AI are in the voice of the personal trainer. It should be noted that in other embodiments, when a personal trainer or 3^{rd} parties goals for the user conflict with the AI goals for the users, the user and/or the personal trainer or 3^{rd} party may have the option to allow the personal trainer or 3^{rd} parties goals for the user to override the AIs goals for the users.

Based on data from a motion sensor (e.g., gyroscope, accelerometer, altimeter, infrared, etc.) of the metric device, the user can be prompted (via his/her mobile device) to slow down or speed up workout repetitions (a.k.a., reps) to achieve maximum performance. For example, speed, acceleration, and/or velocity data can be sent from the metric device to the user's mobile device in real-time or near real-time as the user is working out. Based on the speed, acceleration, and/or velocity data, the mobile device can perform a comparison of whether the user is achieving his/her workout goals. If the user is not achieving his/her workout goals, then the mobile device can prompt the user to change his/her actions (e.g., speed up, slow down, keep a steady state, etc.) to achieve his/her goals. For example, one goal may comprise holding the workout equipment at a specific location for a specific amount of time (e.g., holding the barbell at its max height for three seconds during a bench press exercise). The metric data can be sent to the third party from the user's mobile device and/or the metric device and stored at a server device and then pulled down by the third-party's mobile device. The user can also be prompted from the third-party device (via communication from the third-party's mobile device to the user's mobile device) based on metric data that has been received by the third-party.

For example, the system can prompt the user to go to the twenty-five pound dumbbells to perform three sets of curls on each arm, next go to the bench press to perform three sets of bench pressing one hundred thirty-five pounds, and then go to the lateral pull-down machine to perform three sets of lateral pull-downs at one hundred fifty-five pounds. Additionally, the user can input (via his/her mobile device) a goal to increase his/her pectoral muscles by twenty percent within two months, and the system can automatically generate an exercise regimen, over a two-month span, for the user based on his/her goals. Alternatively, a third-party can generate the exercise regimen and provide it for access by the user's mobile device and/or the metric devices. The exercise regimen can comprise detailed instructions on what exercises to perform on specific days and times as well as what nutrition the user will require based on the aforementioned received inputs. The user prompts can be displayed on the user's mobile device and/or transmitted audibly to the user from the mobile device.

It should be noted that the system can also provide limited data to nearby mobile devices based on a current user's workout session. Since the metric device and/or the system can store the details of the user's exercise plan, the device and/or the system can keep track of where the user is within his/her workout plan. For instance, if a user has received an audible indication to perform three sets of one hundred thirty-five pounds on the lateral pull-down machine at ten repetitions a piece, and the user begins performing this exercise, the metric device and/or the system can determine where the user is within the workout (e.g., on the 5^{th} repetition of the second set), an approximate end time, and/or generate approximate end time data. The approximate end time can also be based on an average that the user has attained by using the metric devices in conjunction with the lateral pull-down machine over a period of various workout sessions. Therefore, the metric devices and/or the system can send time and/or approximation data to nearby mobile devices to indicate to nearby gym-goers when the lateral pull-down machine should be available for use by them after the approximated end time.

Based on this data, the metric devices and/or the system can accept a reservation for next use from a gym-goer that received the time and/or approximation data. Additionally, the metric devices and/or the system can generate wait times for a first user to rest in between sets and/or generate wait times to allow a second user to work in with the first user. For example, the system can generate an audible message to be sent to the user's mobile device to alert the user to rest sixty seconds before beginning his next set of repetitions on the lateral pull-down machine. During that same sixty seconds, the system can send audible data to the user's workout partner to begin his workout, which should take less than sixty seconds, while the user is resting, thereby increasing workout efficiency for both the user and the user's workout partner. This type of interplay can be generated by a partner mode, which can be selected on the user's mobile device and/or the user's partner's mobile device. Individual modes and group modes (as discussed later with regards to gamification) can also be selected to determine the type of workout a user would like to facilitate.

In a further embodiment, the system can help users to locate a potential workout partner. For instance, if a first user selects the locate partner mode, then his/her mobile device can locate a second user who has also selected the locate partner mode. Additionally, the system can present the first user with a list of other users who have selected partner mode for a specific day and/or time. Within the locate partner mode, physical fitness biographies, biographical data, exercise regimens, strength, endurance, and/or body type, etc. can be displayed so that the first user can select a partner to workout with that is similarly suited. For instance, if the first user wants to work on pectoral muscles, triceps, and biceps today, then the system might return other users, who want to work on at least some of those muscle groups, to prompt a match. Consequently, if both users agree to proceed, then they can be prompted by their respective mobile devices to work out together leveraging the partner mode interplay as noted above. The system can make suggestions and/or randomly select a workout partner based on data that shows some type of commonality between users. The agreement to proceed is important so that a user who can bench press between two hundred to two hundred and fifty pounds is not inadvertently paired with another user who can only bench press one hundred pounds. If these two users were paired together, then they would have to rack and un-rack the weights for each set to accommodate each user, which can result in an inefficient workout.

Alternatively, the first user and/or the system might select a second user based on a training-type platform. For instance, the first user might want to have larger calf muscles, therefore the first user can search for other users, via the mobile app, that have larger calf muscles. The search can be based on pictures of the other users calf muscles, data from machines and/or metric devices that the other users have utilized to exercise their calf muscles, profile data, frequency of use of machines and metric devices that were used for exercising calf muscles, or the like. It should be noted that this scenario is not limited to calf muscles and can apply to any other body part such as abs, triceps, biceps, glutes, etc. Thus, the system can scan for other users who attend the same gym and/or are nearby who have strong calf muscles and ask the other users if he/she would be willing to partner with the first user for a calf muscle workout.

In another embodiment, an ad hoc trainer mode can be utilized. For example, the first user can search for a trainer that he/she would like to utilize to train his/her abdominal muscles. The search results can yield pictures of the trainers abs, pictures of the trainer's clients abs, data based on the metric device use for the trainers abdominal workouts, comments from previous and/or current clients, ratings from the previous and/or current clients, cost per session, diet information, nutrition information, location, hours of availability, body part focus areas of the trainer, sports based focused areas (e.g., weight training for swimming, baseball, basketball, football, la cross, tennis, etc.), etc. The first user can schedule a session with the trainer for later and/or if the first user and the trainer are currently in the gym (based on a determination the first user and the trainers mobile devices are within a defined proximity of each other), the first user can send a message to the trainer to request to workout with him/her immediately in the same manner as the partner mode. One difference between the partner mode and the trainer mode is that the trainer can request to be paid for this as a service through the mobile application. Thus, the first user can use a credit card associated with the mobile application to pay the trainer for the session.

In another embodiment, it should be noted that a user's workout plan can be altered by the availability of certain machines. For example, a first user's workout plan may have him perform a set of lateral pull-downs, a set of chest bench presses, and a set of curls using dumbbells in that specific order. However, when the first user walks into the gym, a second user is currently using the lateral pull-down machine and there is an approximate estimated ten-minute wait time until the second user will complete his workout on the lateral pull-down machine. Consequently, because the system recognizes that the first user will have to wait ten minutes before beginning his workout, the system can check to see if any of the other exercise stations are available (e.g., the bench press, dumbbells) and then direct the first user to the bench press to begin his workout instead of waiting an additional ten minutes. The system can also take into consideration whether the first user's workout might be less efficient or productive based on the reordering of the exercises to be performed. For example, if performing the bench press prior to the lateral pull-down is counter-productive, then the system might suggest that the first user begin with the dumbbells, then the lateral pull-down, and then the bench press to avoid the overall ten-minute wait. It should also be noted that metric devices attached to various exercise machines can form a network of metric devices that can communicate with each other to generate usage data and assist in redirecting users accordingly.

The metric devices can control the resistance or weight to be lifted for exercise machines. Based on user inputs (e.g., third-party inputs such as from a personal trainer, algorithms, etc.), the metric devices can be configured to adjust a weight and/or resistance associated with the exercise equipment. For instance, a personal trainer who is geographically remote to the metric devices can send an input (over a wireless network) to the metric devices to increase or decrease the weight of a lateral pull-down machine that the user is using or is about to use. Alternatively, the input can be sent to the mobile device of the user and communicated to the user via a text message or an audible signal. Accordingly, the user can then adjust the weight of the lateral pull-down manually. Consequently, when the repetition is completed by the user, completion data representative of the lateral pull-down exercise being completed can be generated by the metric device and sent to the mobile device and/or the remote server for access by the personal trainer and/or another third-party.

The metric device's wireless communication can leverage location-based services based on a defined distance. For instance, the metric device of a lateral pull-down machine can be set to communicate with mobile devices within a defined distance/radius (e.g., one foot) or zone of the lateral pull-down machine. This can prevent the metric device from communicating with other mobile devices that are not within the radius or zone of the lateral pull-down machine, thereby allowing the metric device to generate metric data for the current user (within the zone) of the lateral pull-down machine. Additionally, by zoning the metric device's wireless communication, the system can prevent the metric device from prompting a user to do an exercise when the user is not within the zone of the exercise equipment. The zone can be defined by the metric device's location and/or the exercise equipment location. Upon detection of a user's mobile device entering the zone, the system can also adjust the weight and/or resistance of an exercise machine according to a defined value. The defined value can come from a third-party device, a preset value from the user's mobile device, and/or based on a generalized workout regimen. For example, in response to a user's mobile device entering a zone associated with a leg press machine, the metric device can facilitate adjusting the weight and/or resistance of the leg press machine to a value set by a trainer remotely.

To prevent false positives and/or false negatives, the exercise equipment, the metric device, and/or the mobile device can indicate which metric device of multiple metric devices should be used in close proximity situations. For example, if the zones of two metric devices associated with two different exercise equipment overlap, it can be difficult to pinpoint which exercise equipment the user is planning on using or actually is using. Thus, based on the two zones overlapping, the user can see and/or hear an indication of such on his/her mobile device and be prompted to select (via the mobile device and/or the metric device) which metric device and/or exercise equipment he/she intends to use. Another indicator of which exercise equipment a user intends to use can be based on additional data from pressure sensors in a seat and/or pads associated with a specific exercise equipment. Thus, when the user is in contact with the seat or pad (e.g., exerting pressure on the pressure sensors), this can confirm the exercise device that the user intends to use. Consequently, a two-prong verification process can be achieved: 1) mobile device is in proximity to the metric device, and 2) a pressure sensor is sensing pressure. In the scenario where workout partners mobile devices are both within the zone and the pressure sensor is sensing pressure from at least one of the workout partners, the mobile devices can request a confirmation of which workout partner is actually initiating the pressure on the pressure sensor. Alternatively, in the case where two metric device zones associated with two exercise equipment are overlapping and the pressure sensors on both exercise equipment are sensing pressure, then the users' mobile devices can prompt the user to indicate (e.g., via the mobile device screen and/or audibly), which exercise machine the user is using. It should also be understood that in scenarios where the metric device is built into the exercise equipment, the exercise equipment itself can comprises a display screen to interact with the users and allow the users to confirm usage of the exercise machine.

In an additional embodiment, the profile of the user can be updated with each workout session and a network of users with user profiles can allow their respective profiles to be seen by the other users. A gamification component of the system can pit one group of users against another group of users, or a first user against a second user, by displaying the first user's exercise metric data to the second user, or group of users, and prompting the second user to exercise. For example, the system can send a notification to the mobile device of the second user saying, “The first user lifted one hundred thirty-five pounds today and burned three hundred calories, what did you do?” Additionally, the gamification component can determine if the group of users have all met their workout goals for the day and provide an indication to the group of users when they have met their goals and/or encourage group members to meet their goals if they have not worked out for the day. For example, the system can send a notification to mobile devices of the group of users saying, “Only three out of the five of you have gone to the gym today, when are the rest of you going?” The system can also specifically indicate which group members have not met their goals and allow the other group members to encourage them to meet their goals.

In certain instances, exercise machines can perform multiple exercises for a user. For instance, the leg adduction/abduction machine is one exercise machine that performs two different exercises. Leg adduction can exercise the inner thighs while leg abduction can exercise the outer thighs. Therefore, the metric devices can be configured to determine which exercise is being performed and which exercise to generate and/or receive data for. Additionally, the system can adjust a user prompt to alert the user to perform leg adduction or perform leg abduction in accordance with a workout regimen and/or in accordance with a directive received from a third-party such as a personal trainer.

When a machine is multi-functional or a variety of workouts can be performed by a free weight, the system can allow the user to select a particular workout via voice or the mobile device. Alternatively, the system can ask the user which workout he/she would like to perform and receive an indication from the user (e.g., via voice or the display screen of the mobile device) as to which workout he/she would like to perform. Alternatively, the gyroscope accelerometer data can be used to predict/indicate which exercise a user is currently performing based on defined motion tolerances that have previously been associated to be representative of specific exercises. For example, motion data from a disk on a dumbbell can generate a specific gyroscope and/or accelerometer pattern such that data from the gyroscope and/or accelerometer can be compared to previous gyroscope and/or accelerometer data to indicate that the user is currently performing a specific exercise (e.g., barbell curl, chest press, tricep extension, or the like). The system can also suggest, based on a third-party input (e.g., from a personal trainer), a goal for the user and/or the user's profile data, which workout to perform, and display the motions visually on the mobile device to perform the suggested workout. If a specific workout is selected via the voice of the user, the system can ask the user to confirm (prior to data being exchanged between the metric device and the mobile device) to prevent false positives or false negatives in case the mobile device receives audible data from another person. It should be noted that this confirmation system can be performed in conjunction with any of the audible features of this system and/or any of the display screen features as well. For exercise equipment, there can be a selector (button) to determine which exercise is being performed or placing the exercise equipment in a certain position can indicate which exercise is about to be performed.

In another embodiment, the metric device can be adapted to charge its battery wirelessly so that the metric device does not have to be removed from the weightlifting equipment (e.g., barbell, dumbbell, leg press etc.) or exercise machines to be charged. The metric device can be wirelessly charged via wireless power transfer, charging by induction (via an induction component of the metric device), and/or resonant wireless charging. For example, the metric device can be so dimensioned to fit on the inner grip area of a dumbbell (e.g., a disk) without impeding the hand of a user while he/she is using the dumbbell. The disk can also comprise copper contacts and/or wireless charging capabilities such that the disk can be charged while the dumbbell is at rest on a dumbbell rack. Consequently, the dumbbell rack can comprise copper contacts and/or wireless charging capabilities to charge the disks on dumbbells while the dumbbells are racked. Thus, the dumbbell rack can comprise an electrical cord to plug the dumbbell rack into an electrical outlet to provide power to the disks attached to the dumbbells while they are racked.

It should also be noted that the smart system can conserve battery power in various ways. For example, if the dumbbell comprising the disk is racked (e.g., being charged) it can be assumed that the dumbbell is not in use and therefore should not be transmitting data. However, once the dumbbell is removed from the charging rack, it can broadcast its Bluetooth signal such that it connects to the nearest mobile device which is predicted to be the mobile device of the person using the dumbbell that is closest to the dumbbell. Once the user's mobile device is connected to the disk via Bluetooth, other data (e.g., gyroscope, accelerometer, altimeter, weight, disk identification, or the like) can be transmitted to the user's mobile device. The disk can be preprogrammed with a weight that corresponds to the dumbbell for which it is attached such that this weight is sent to the mobile device of the user. If the disk is changed or moved to another dumbbell, the programmed weight can be modified (via the administrator account mentioned above) to reflect the weight of the new dumbbell. When a user is using multiple dumbbells, each dumbbell can connect to the user's mobile device independently, or a secondary dumbbell can communicate its data to a primary dumbbell, and the primary dumbbell can communicate its data and the data from the secondary dumbbell to the mobile device of the user.

Different exercise machines may be calibrated differently. Therefore, a self-assessment of exercise machines and their performance can be communicated to the mobile device, via the metric device, to facilitate normalizing a user's workout metrics for the exercise machines. For example, a first pectoral fly machine may exhibit a different resistance using a forty-five-pound weight than a second pectoral fly machine using a forty-five-pound weight. Therefore, as a user uses the first pectoral fly machine, his/her metrics (e.g., acceleration, velocity, etc.) can be different than when the user uses the second pectoral fly machine. In one embodiment, the system can use the metrics from the exercise machine that the user uses the most to generate a baseline of metrics and then normalize metrics from other exercise machines based on the metrics from the exercise machine the user uses the most. For example, if the user uses the first exercise machine (e.g., at a home gym) the most, and then uses the second exercise machine (e.g., at a visiting gym), then the system can normalize the metrics from the second exercise machine at the visiting gym against the baseline of the metrics from the first exercise machine of the home gym. This process can prevent an un-calibrated exercise machine from providing faulty data associated with a user's workout.

If the system recognizes that it is a user's first time using a specific exercise equipment (e.g., the mobile device has never connected with a metric device of a specific exercise equipment and/or a previous use of the exercise equipment and/or the metric device is not listed in the users profile), the metric device can communicate with the mobile device to have a user calibrate his/her workout settings, during a pre-workout, for using the exercise equipment. The calibration can comprise prompting the user to step through several reps of using the exercise equipment (e.g., minimum reps, average reps, maximum reps, minimum weight, average weight, maximum weight, rep time, rest time, etc.) to determine the user's baseline fitness and normalize the user's suggested workouts based on metrics associated with the user's fitness, the user's workout goals, and/or data received from a third-party.

In another embodiment, during the pre-workout, the normalization can be based on a selected workout type (e.g., add muscle, burn fat, bulk-up, tone-up, etc.) by the user. For example, if a user wants to tone-up, then generally more repetitions with less weight can accomplish this goal. Therefore, the system might prompt the user to max out his/her repetitions at a particular weight to assess at what weight/resistance the user should begin his/her actual workout. For this particular example, during the pre-workout, the system can determine what weight/resistance allows the user to achieve between twelve to fifteen repetitions and use that weight as a beginning point for the user during his/her actual workout session. Thereafter, the system can return a regimen to be used at a later time for the user's actual workout (e.g., three sets of twelve repetitions of one hundred pounds).

Pressure sensors can also be used to indicate the weight of a barbell and/or dumbbell. For instance, a twenty-pound dumbbell can exert a different range of pressure in the hand of a user, than a twenty-five pound dumbbell. Likewise, ninety pounds of weight on a barbell will exert a different pressure on a user's hand than fifty pounds of weight on the same barbell. Therefore, pressure sensors can be placed on the dumbbell and/or barbells bar (e.g., grip area) so that the pressure generated as the bar is being pressed against the user's hand (or neck of the user if squatting) can indicate how much weight is on the barbell and/or the dumbbell. This data can then be sent to the mobile device to generate additional data (e.g., caloric expenditure, muscle strain, resistance, etc.) that can be stored in a database or in a user's mobile profile for immediate access and/or access at a later time.

The metric device system can also comprise various security features. For example, a mobile device can be paired with the metric device through Bluetooth and/or a mobile application. To access certain features of the metric device (e.g., weight selector pin), the user can be required and/or prompted to provide a valid credit card associated with the mobile application. For example, in order to download and/or utilize the mobile application, the user can be prompted to provide a valid credit card number and/or utilize a stored valid credit card number within the user's mobile application download account. When a mobile device is paired to the metric device via Bluetooth, the GPS of the mobile device can reveal the location of a user of the mobile device, thereby determining that the metric device is in the same location as the user's mobile device. Because the metric device can also have a predetermined location associated with it, the mobile device's GPS location can be compared to the predetermined location of the metric device to see if they are consistent. If the locations match, then it can be assumed that the metric device is in a proper location.

However, in the case of an inconsistency, it can be assumed that the metric device is not currently at the predetermined location (e.g., the metric device is lost or has been stolen). In this case, if there is a wireless connection between the metric device and the mobile phone outside of the predetermined location (e.g., the user is trying to utilize the metric device outside of its predetermined location), the credit card provided by the user can be charged for the cost of the metric device. The latest GPS coordinates of the most recent connection between the metric device and the mobile device can also sent to a third-party for analysis. Furthermore, if the user of the mobile device has been charged, the user can receive a prompt that the amount the user was charged (or a portion thereof) can be refunded if the user returns the metric device to its predetermined location or a designated location provided by the third-party. Other embodiments of this disclosure can comprise a GPS tracker in-built in the metric device itself to facilitate the same or similar processes mentioned above. In this scenario, the metric device can send an indication that it is outside of the predetermined location upon the metric device leaving the predetermined location.

In another embodiment, the metric device can comprise an RFID tag and/or a near field communication tag to prevent theft. For example, as seen in department stores, when someone tries to walk out of the store with goods that have not been paid for, the tag readers at the door can determine that the tags are still active when the goods are brought within a distance of the tag readers. Thus, an alarm will sound. Similarly, RFID readers installed at the entrance/exit of an exercise facility can alert the appropriate personnel when the metric device (or the RFID tag of a metric device) is within a certain distance from the tag reader, thereby preventing theft.

Some exercise machines can be capable of performing multiple exercise (e.g., dual-use machine, tri-use machines, etc.). An example of this would be an exercise machine that can be adjusted to perform a hip adductor exercise or a hip abductor exercise. Some embodiments of the mobile application can display the different exercises that can be performed on a dual-use machine. Thus, a user can select which exercise the user is performing by allowing the user to toggle between the different exercises displayed by the mobile application (e.g., hip adductor exercise or hip abductor exercise). Consequently, although data from the weight selector pin for the hip adductor or the hip abductor would be the same, the user can select the appropriate exercise to correspond to the data within his/her mobile app. In other embodiments, the user can communicate which exercise the user is performing verbally via a microphone of the mobile device and/or a microphone of the metric device. This can then allow the data received from with the metric device to be associated with a particular exercise. In another embodiment, the data received from the metric device can be associated with a specific exercise via an edit feature found within the mobile application. Therefore, a user can add this information later and/or change the name of a first exercise to now reflect the name of a second exercise associated with a dual-use machine. In yet another embodiment, a multi-exercise machine can comprise RFID tags that correspond to each exercise. For example, the hip adductor exercise can be selected by near field communication between the metric device, mobile phone, and/or other devices (e.g., wearable device, Apple Watch®, or the like) in communication with the mobile device or metric device and the RFID tag. When the user is ready to perform the hip abductor exercise, he/she can wave his/her Apple Watch® near the hip abductor RFID tag, which can communicate the hip abductor exercise to the users mobile phone such that the impending exercise data is logged against the hip abductor exercise and not the hip adductor exercise. Because different exercise machines have varying weight stack heights, the RFID tag can also be used to log the current exercise against height values (e.g., from the infrared sensor) that have been stored based on the height of the metric device at each weight stack such that weight detection algorithm provides the proper value.

Each metric device can comprise a unique identification (e.g., Bluetooth signal name, given label, given name, etc.) and/or send a unique signal associated with it. The metric device can also be named after the exercise machine for which it is being used (e.g., bicep curl machine). However, weight selector pins can be interchangeable with various exercise equipment machines, and thus a proper naming convention can be used to determine which weight selector pin is actually associated with a specific exercise machine. Therefore, name spoofing can be utilized to keep data consistent. The name spoofing can be performed by a mobile device that has administrator privileges via a mobile application. For example, if a unique ID of a weight selector pin is device 123, this data can be stored in a database via the mobile device. The mobile device can be capable of associating the device 123 with a specific exercise machine (e.g., bicep curl machine). For example, if the device 123 pairs to the mobile device via Bluetooth, the mobile device can know that it is communicating with the device 123. Thus, a mobile device with administrator privileges can select and/or input that the device 123 is to be associated with the bicep curl machine (e.g., new name) and store this data in a database. Consequently, another mobile device (utilized by an end-user) that subsequently pairs to the device 123 via Bluetooth to receive and/or assess data streaming from the device 123 can receive the new name (e.g., bicep curl machine) of the device 123 from the database, and therefore associate the data being received from the device 123 with the bicep curl machine name.

Consequently, the spoofing can automatically let the other mobile device (e.g., end-user mobile device) know that the user is using the bicep curl machine and the data being streamed from that particular session with device 123 is associated with the bicep curl machine. The data can then be converted into machine repetitions, sets, rest time, etc., associated with the bicep curl machine in or near real-time.

At some point in time, the device 123 can be swapped out for another weight selector pin. For instance, if a battery of the device 123 is dead or dying, a replacement weight selector pin (e.g., device 200) can be utilized. If the weight selector pin (e.g., device 200) is to be utilized with the bicep curl machine, the mobile device can utilize the aforementioned spoofing process to now associate the device 200 with the bicep curl machine and/or disassociate the device 123 with the bicep curl machine via the administrator account. It should be noted that in some embodiments, association of a new device with the bicep curl machine can automatically disassociate a previously spoofed device. However, there can be multiple ways that the device 200 is associated with the bicep curl machine. For instance, the unique device 200 ID can already be stored in the database that houses data representative of the device 123 being associated with the bicep curl machine. In this the scenario, a user of the mobile device can utilize the mobile application to now associate the device 200 with the bicep curl machine. Thus, when a Bluetooth connection occurs between the device 200 and another mobile device, the other mobile device can receive data from the database indicating that the device 200 is now associated with the bicep curl machine.

Alternatively, as previously discussed (with regards to device 123), the device 200 can be paired to the mobile device via Bluetooth for the mobile device to receive the unique ID (e.g., device 200) associated therewith, and then the mobile application can allow the device 200 to be associated with the bicep curl machine. In yet another embodiment, the mobile application can prompt the administrator to perform a series of spoofing steps to automatically transfer the bicep curl association from the device 123 to the device 200. For example, if the device 123 is already associated with the bicep curl machine, when the administrator account is opened, the mobile application can prompt the administrator to next wirelessly pair (e.g., via Bluetooth) the weight selector pin for which he/she would like to transfer the bicep curl name to. Thus, when the mobile device and the weight selector pin (e.g., device 200) are paired, the mobile device can receive the ID (e.g., device 200) and then automatically associate the bicep curl machine name with the device 200 in response to the device 200 being paired to the mobile device based on the prompt.

In an alternative embodiment, prior to the aforementioned process, the mobile application can first prompt the administrator to wirelessly pair (e.g., via Bluetooth) with the weight selector pin (e.g., device 123) for which the name is to be taken (e.g., bicep curl machine). The mobile device can receive the weight selector pin ID (e.g., device 123) from the weight selector pin, receive the associated name (e.g., bicep curl machine) from the database, and can then request the administrator to wirelessly pair (e.g., via Bluetooth) the weight selector pin (e.g., device 200) for which he/she would like to transfer the bicep curl name to. It should be noted that any machine name can be associated with any weight selector pin. It should also be noted that in alternative embodiments, multiple weight selector pins and their respective IDs can be associated with a specific exercise machine. For example, any weight selector pin that has an ID ending in “5” (e.g., device 105, device 205, device 305, etc.) can be associated with the bicep curl machine, such that any connection of another mobile device (e.g., end-user mobile device) with device 105, device 205, or device 305, via Bluetooth, can correspond to the bicep curl machine. It should also be noted that the administrator feature can reside on the end-user mobile application and be accessible by some security feature (e.g., password, facial recognition, fingerprint, voice, biometric, etc.).

Each metric device can communicate its battery life to the mobile phone to which it is paired. This data can then be sent from the mobile phone to a server device to facilitate timely replacement of the metric devices that experience low or dead battery life. For example, for a specific location, multiple threshold values can be set such that when 20% (or a certain number) of the metric devices at that location have a battery life of less than 15%, then an alert can be sent to a third party. The alert can prompt the 3^{rd} party to bring additional fully charged metric devices to the location such that end-users do not experience a lapse in service. Based on the aforementioned name spoofing processes, the 3^{rd} party can associate the fully charged metric devices with the machine names for which he/she will be replacing the metric devices with low or dead battery life. The third party can be paid on per metric device or per trip model. For example, the third party can be responsible for charging the metric devices that he/she picks up and distributing them a predetermined location and/or at a facility that needs newly charged metric devices.

One metric that can be collected from a user's workout can be the number of repetitions (“reps”) of the exercise movements performed. A rep can be calculated by checking whether the user has incorporated a full range of motion as defined by an algorithm utilizing infrared sensor (e.g., time-of-flight sensor) data and/or a force curve associated with accelerometer data. A user's full range of motion can be dependent on factors such as the type of exercise equipment and/or physical characteristics of the user (e.g., length of arms, length of legs, etc.), which can be manually input into the mobile application as user profile data. In another embodiment, a biometric scan of a user can measure the physical characteristics of the user and the biometric scan data can be used to automatically generate and/or populate the user's profile or a portion thereof. Based on a travel distance, (e.g., minimum travel distance, maximum travel distance, and/or threshold distance) and/or acceleration curve associated with the metric device, (e.g., weight selector pin, a barbell sleeve device, a dumbbell disk, and/or a plate loaded machine bumper), a user can be determined to have completed a full rep.

However, if the weight selector pin does not travel a specific threshold distance, or come back down within another specific threshold distance, it can be an indicator that the user has not performed a complete rep. Likewise, if the acceleration curve does not meet a threshold value, then it can be an indicator that a rep has not been completed. If the user has not completed a full rep, then the rep counter can be prevented from incrementing and/or the user can be provided with an indication (e.g., text, audible, haptic feedback, etc.) that a proper rep has not been completed. The shortcoming data (e.g., difference between the distance travelled and the threshold for a complete rep and/or the difference between the acceleration and a threshold value) associated with the user's failed rep, can be calculated to provide the user with additional post-workout information such that the user can work to correct his/her form deficiencies. In some scenarios, the user might not perform a proper rep because the user's muscles are fatigued. In such a scenario, as the user is attempting to meet the threshold distance and/or acceleration to complete the rep, the user may experience a stall effect, whereby the users muscles fatigue and the distance of the weight selector pin travel and/or the acceleration experienced by the metric device is stalled on the way to the threshold. Although the user might not receive a rep count because the threshold value is not met, the time that user experiences the stall effect can be utilized to provide the user with additional post-workout data. For example, as compared to other workout sessions, the data can indicate if the stall time was longer, which can indicate increased strength/endurance of the user. Or if the travel distance of the metric device was closer to the threshold distance and/or the threshold acceleration when compared to previous workout sessions, this can also indicate increased strength/endurance gained by the user. Thus, a premium feature can be made available via the mobile application, whereby a personal trainer can access the user's data to check and see if there is any incremental progress in the user's ability to perform the exercise, and/or provide the user with additional insights and/or guidance.

For proper rep determination, a user should strive to have the metric device cross a top height threshold during the first half of his/her rep and a bottom height threshold during the second half of his/her rep. As the metric device is moving upward during the first half of a rep, rep determination can be based on taking a current height (e.g., distance of the metric device from a reference point based on infrared sensor readings) reading, as the height continues to increase, until the infrared sensor readings provide a decrease in the height reading, which indicates the user has begun the second half of his/her rep (e.g., the metric device is now moving downward). The height of the metric device at the peak (e.g., the highest/greatest value just before the decrease in values) can be used as one of the max height values for the averaging process as noted below. Thus, the peak value can be determined based on a decrease in the current height reading as compared to a previous height reading. Alternatively, as the metric device is moving downward during the second half of a rep, rep determination can be based on taking a current height (e.g., distance of the metric device from a reference point based on infrared sensor readings) reading, as the height continues to decrease, until the infrared sensor readings provide an increase in the height reading, which indicates the user has begun a first half of a new rep (e.g., the metric device is now moving upward again). The height of the metric device at a valley (e.g., the lowest value just before an increase in values) can be used as one of the minimum height values for the averaging process as noted below. Thus, the valley value can be determined based on an increase in the current height reading as compared to a previous height reading.

It should be noted that other sensors can also be used to determine minimum and maximum values for rep determination by using altitude data from an altimeter of the metric device, acceleration data from an accelerometer of the metric device, magnitude data from a magnetometer of the metric device, or the like. For example, in certain instances, the infrared sensor may be out of range during use. In such a case, another sensor (e.g., accelerometer, magnetometer, gyroscope) can be relied upon to help compensate for data where the infrared sensor is out of range. For example, if the infrared sensor goes out of range during use of the metric device, then the accelerometer sensor data can be used to compensate for the lapse of infrared sensor data by determining when the metric device has changed velocity based on determining min and max velocity values generated by the accelerometer and utilizing the aforementioned processes.

In addition, or alternative, to physical attributes of a user and/or the weight equipment, rep determination can also be performed based on an averaging process. For example, an average cycle time, height, and/or acceleration associated with a user's first three reps can be used as a baseline to determine whether the user shall have a rep counted or not. It should be noted that any number of reps can be used to determine the average (e.g., 1, 2, 3, 4, etc.). For example, if a user performs four reps averaging a max height of the metric pin device of 500 millimeters at the top of the rep and a min height of 300 millimeters at the bottom of the rep, then future reps can be counted if the metric pin device height is equal to or greater than 500 millimeters at the top of the rep and is equal to or greater than 300 millimeters at the bottom of the rep. Alternatively, if a user performs four reps averaging a max acceleration of the metric pin device of 2 meters per second squared at the top of the rep and a min acceleration of 1 meter per second squared at the bottom of the rep, then future reps can be counted if the metric pin device acceleration is equal to or greater than 2 meters per second squared at the top of the rep and is equal to or greater than 1 meter per second squared at the bottom of the rep. In some scenarios, the first rep (or a number of preset reps) data can be removed from the averaging equation because the user's body is adjusting to the resistance during the first rep, which may not be an accurate depiction of the overall rep data that should be included in the averaging to determine the threshold. For example, the first rep height (of the metric device 100) may be 300 mm (e.g., the user's body is adapting to the resistance) while the second and third rep heights are 380 mm and 400 mm, respectively. Thus, the first rep data (e.g., 300 mm) can be thrown out as an outlier, and the second and third rep heights can be averaged for an average height value of 390 mm, which is truer to an actual representation of the user's rep span.

The weight selector pin can comprise a two-step verification factor to determine the authenticity of the identity of the user using the weight selector device. For example, in one embodiment, the weight selector pin can comprise a finger-print scanner to facilitate a fingerprint analysis based on a user touching the weight selector pin. The fingerprint analysis can determine that the user has accessed the weight selector pin and then the user can be authenticated as actually having performed a workout by leveraging heart rate data, which can be received from another device (e.g., Apple watch, etc.), to indicate that the user is actually performing the workout and not someone else.

In a partner situation, where only one person's mobile device may be connected to the metric device, there can be a primary user and a secondary user. For instance, the mobile device of the first user (e.g., primary user) can connect to the metric device via Bluetooth when he/she is working out. However, when the second user (e.g., secondary user) begins his/her workout, instead of the first user's mobile device disconnecting from the metric device, to allow the second user's mobile device to connect, the first user's mobile device can receive the data (via Bluetooth) associated with the workout of the second user and then transmit (e.g., via Bluetooth, Wi-Fi, cellular, etc.) that data to the mobile device of the second user and/or send the data to a server device to be sent to the second user. This functionality can be performed based on the users selecting the aforementioned partner mode previously discussed.

In some circumstances, it may be possible to spoof workout data by moving the metric device in a certain motion to generate data. For instance, if a user is trying to replicate the motion of the weight selector pin attached to a weight stack, he/she can move the weight selector pin up and down to simulate repetitions. However, to prevent this from occurring, additional data can be leveraged to indicate when a user is trying to spoof the system. For example, gyroscope data can be used to determine that the weight selector pin is not levelled during the use, and thus, spoofing can be detected, and the system can generate an output detailing such and/or not perform a repetition count. It should be noted that data from other components can also be used to determine spoofing (e.g., if altimeter data indicates that the metric device was too high to have been used properly, etc.). Alternatively, the gyroscope data can be used to ascertain if a user is not performing a proper repetition or is in danger of injuring himself/herself. For example, the gyroscope data can indicate that a barbell in use (or previously in use) is not (or was not) levelled during a bench press exercise, thus indicating that one of the user's arms was not extended as far as his/her other arm. Depending on the gyroscope motion, the gyroscope data can indicate which of the user's arms is stronger than the other.

Consequently, such data can be analyzed and presented to the user via his/her mobile device to indicate to the user that he/she should lower his/her bench press weight to prevent injury and/or to work on strengthening the weaker arm.

The alert can be based on some type of threshold set by the user, 3^{rd}-party, personal trainer, physical therapist, artificial intelligence, etc. For example, an alert can be issued if the user ever applies more than 20% of his/her max lift weight to a barbell. This can be especially important to physical therapists and insurance companies with the user/patient has been instructed to not add additional weights. An alert can also be issued if the weight is uneven on the barbell (e.g., the user has applied 50 pounds on one side of the barbell and 60 pounds on the other side of the barbell). In some scenarios this can be determined by one metric device whereas in other scenarios there may be two metric devices (one on each side of the barbell). A similar alert system can be applied to selectorized machines that utilize the weight selector pin when the user has inserted the pin into a weight stack that is too heavy for them, it can be applied to the use of dumbbells when the user has picked up a dumbbell that is too heavy for them, and it can also be applied to plate loaded machines when the pressure sensors in the bumper indicate that the weight is too heavy for the user. It should also be noted that alerts can be issued, when the AI or 3^{rd} party has listed a workout weight within the mobile app and the selected weight by the user is different. Confirmation alerts can also be generated when a correct weight has been selected.

In some embodiments, a gromet with an increasing external slope can be used to place around the pin of the weight selector pin device to increase the friction as the pin is inserted into a weight stack. Alternatively, sleeve like adapters can be used to convert smaller diameter pins to larger diameters pins such that the smaller diameter pins can fit into larger diameter holes of weights and increase the friction between the two. This can facilitate stability of the weight selector pin device as it experiences motion during use. Additionally, other metrics from the weight selector pin can be used to determine the orientation of the pin. For example, a weight selector pin can be configured to operate with any pin-based exercise machines, such that correct data is provided. Various exercise machines have different heights for their weight stacks and each weight stack can vary by standard weight intervals (e.g., 10 lbs, 15 lbs, or 20 lbs). However, each weight stack is generally one inch in thickness. Thus, if the height of a bottom weight of a weight stack of particular machine is known, the weight (e.g., resistance experienced by a user of the machine) of the bottom weight of the weight stack is known, and the weight intervals of the weights of the weight stack is known, then the infrared sensor can determine which height and weight corresponds to each weight of the weight stack.

For example, if the bottom weight stack is 4 inches above the ground, the bottom weight stack is 250 lbs, and there is a 15 lbs interval between each weight stack, then the system can determine that when the weight selector pin is placed in a weight stack 5 inches from the ground, that the user has placed the weight selector pin in the weight of the weight stack that is representative of 235 lbs. This process can be facilitated by a user placing the weight selector pin in the bottom weight of the weight stack (wherein the infrared sensor generates a distance value), the user enters the pounds of the bottom weight, and the pound intervals between the weights into his/her mobile device. The user can be prompted via their mobile device to perform each step via a configuration process. It should be noted that this same process can be performed by placing the weight selector pin in the top weight of the weight stack, entering the associated pounds of the top weight, and entering the pound intervals between the weights of the weight stack. In some embodiments, the user can be instructed to perform a calibration procedure at the bottom weight and the top weight of the weight stack such that variability in the height data can be synthesized. In other embodiments, the user may be asked to place the weight selector pin in each weight stack to measure the location of the weight selector pin at each weight stack to account for any variability in the infrared sensor data. In use, the current height values provided by the infrared sensor (e.g., time of flight sensor) can be compared against the previous height values associated with each weight stack whether it be from the weight selector pin being place in each weight stack or whether the weight selector pin was only placed in the top and/or bottom weight stacks to have the height values propagated accordingly.

The user can also enter the machine name (e.g., bicep curl machine) that the weight selector pin is now calibrated for in their mobile device. This data can be sent to a server device to be stored, such that when a new mobile device connects to the weight selector pin, the new mobile device can receive weight stack data (e.g., height and pounds of each weight of the weight stack) corresponding to the machine name data from the server device, based on the weight selector pin being previously configured for the bicep curl machine. As mentioned previously, in scenarios where multiple exercises can be performed on one machine, RFID near field communication can be utilized the determine which workout the user is performing. Thus, a single calibration procedure at one machine, can be used for multiple exercises at the same machine because the same weight stack is used to perform the various exercises.

Because the infrared sensor can generate distance data to determine what weight has been selected, if the weight selector pin device is not properly oriented, false values that are not representative of an actual distance of the infrared sensor from the ground can be generated, which can disrupt the configuration/calibration data. However, gyroscope and/or accelerometer data from the weight selector pin can be used to mitigate this occurrence. It should be noted that an angle/orientation of the weight selector pin can be determined by computing the arcsine of accelerometer data. For example, using the Pythagorean theorem, a proper linear distance can be determined even if the infrared sensor of the weight selector pin device is not properly oriented (e.g., the weight selector pin is not parallel to the surface of impact of the beam form the infrared sensor) such that the beam from the infrared sensor is hitting the surface at a 90 degree angle. The gyroscope and/or accelerometer data can determine if the weight selector pin is oriented such that the beam is not hitting the surface at a 90-degree angle. Thus, if the gyroscope and/or accelerometer data indicates that the beam cannot hit the surface at a 90 degree angle, the gyroscope and/or accelerometer can generate data representative of the angle at which the weight selector pin is projecting the beam with respect to the beam from the infrared sensor (e.g., there is a 20 degree angle between the infrared beam at its normal position where it hits the surface at 90 degrees and the current position of the infrared beam) when the weight selector pin is orientated at a normal levelled position (e.g., there is a 90 degree angle between the beam and where the beam impacts the surface). Based on this information, an audible signal or text message can be generated to alert the user to level the pin such that the beam angle of incidence with the surface is closer to 90 degrees.

Consequently, since the two angles are known, the third angle can be calculated. Since the length of the hypotenuse (e.g., the distance of the beam when unlevelled) is known and the two angles are known (e.g., the angle at an unlevelled position and the 90 degree angle at the levelled position), then the third angle can be calculated (e.g., third angle=180−90−(the angle at an unlevelled position)). If the 3 angles and the length of 1 side (e.g., the hypotenuse) is known, then the length of the other two sides of the triangle can be calculated. For example, if the sides are a, b and the hypotenuse is c (opposite angle A), and the angles are A, B and C, then Sin A=a/c, so a=c (Sin A). Also, Cos A=b/c, so b=c (Cos A). Thus, when the weight selector pin is unlevelled, the distance, of the predicted infrared sensor from the surface when the selector pin is levelled, can be calculated based on the gyroscope and/or accelerometer data and the distance that the infrared sensor is generating in its unlevelled state.

Under the Pythagorean theorem, a²+b²=c². For example, if the weight selector pin is placed in the 250 lbs weight stack at a levelled position, the beam distance can yield 1600 mm. If we take the distance of the infrared beam at normal (e.g., levelled position) to be b², and we can take c² (e.g., the hypotenuse) to be the distance of the infrared beam at an unlevelled position of the weight selector pin, then we can correlate the two such that the distance of the infrared beam at the unlevelled position still corresponds to the proper weight of 250 lbs. Thus, taking 30²+40²=50², we know that an unlevelled distance of 2500 mm for the hypotenuse (e.g., c²) can correspond to the levelled distance of 1600 mm. Consequently, if the weight selector pin is unlevelled and the distance data yields 2500 mm, we know that the 2500 mm corresponds to the 1600 mm, which can correspond to the weight of 250 lbs, rather than taking the 2500 mm distance to correspond to a lighter weight (e.g., 220 lbs) as it would if the weight selector pin were levelled at the 220 lbs weight of the weight stack and the distance perceived based on the infrared beam is 2500 mm (e.g., greater distances correspond to lower weights as the weight selector pin is moved up the weight stack).

During a workout flow, a user generally inserts a weight selector pin when he/she begins his/her workout and/or when they want to modify the resistance they are using during the workout. Thus, these intricacies can be taken into account during a data intake mode of the system, wherein the user is performing his/her workout without any guidance from a virtual component. However, the way that the user interacts with the weight selector pin can determine how data from the pin is manipulated via the user's mobile device.

Without any indication from the user, the system can ascertain whether a user has begun a new set or completed a set using analytics and data from the weight selector pin. For instance, the motion of the weight selector pin in the Z plane can initiate a new set (e.g., the user has pulled the weight selector pin out of a weight stack and/or the user has pushed the weight selector pin into a weight stack) because generally, the user does not move the weight selector pin unless they are beginning a new set. However, the scenario where the user does not change the weight and wants to begin a new set can be taken into account based on a time function (e.g., if no motion of the weight selector pin in X seconds, a new set can increment using same weight on the users mobile device). This scenario indicates that a user could be working out with the 30 lbs weight stack, rest for 30 seconds (which is greater than X seconds), and then continue working out with the 30 lbs weight stack. The set can be incremented once the user begins a new rep after the 30 seconds as lapsed. However, if Z-axis motion occurs (e.g., the user pulls the pin out and reinserts) after the new set has incremented, the incremented set remains the same and the rep counter can increase with each rep performed. This scenario indicates that the user waited until after his/her rest time was up (which is longer than the X seconds) to adjust the weight selector pin to a new weight stack and should only receive one set increment and not two.

Thus, at 0 reps, set 1, the system can remain on set 1 until there is at least 1 rep as determined by the up/down motion of the weight selector pin. After 1 rep, and there is Z-axis motion (e.g., the user has pulled out and/or pushed in the weight selector pin), it can be assumed that the user wants to begin set 2 because they have moved the pin. However, there are at least two exceptions to that rule. The first is that it is possible that the user selected too much or too little weight and needs to adjust the weight selector pin but wants to remain on the first set. For this scenario, a threshold can be used such that any number of reps greater than the threshold number of reps (e.g., >3 reps) should indicate that the user has selected the correct weight and any Z-axis motion thereafter initiates a set increment. The second exception is when the user wants to begin a new set but remain on the same weight. For this scenario, as indicated above, the user's rest time can initiate the set rollover. For example, the mobile application can display, set 1, reps 10. If no motion on the Z or Y-axis for >10 secs, set=set+1, and reps=1 after Y-axis motion cycle (e.g., up down motion required for 1 rep). So, if there is no motion on the Z or Y-axis for >10 seconds, but then there is motion on the Y-axis, then the rep counter generate the number 1 to indicate the first rep of a new set and the set counter can increase after the Y-axis motion cycle (e.g., up down motion required for 1 rep). It should be noted that any of the sensor data communicated to the mobile device via various sensors of the metric device can be manipulated and displayed visually via the mobile device to produce such results as reps, sets, rest time, etc. This data can also be communicated audibly via a speaker of the mobile device.

It should also be noted that an artificial intelligence (AI) component can facilitate automating one or more features in accordance with the disclosed aspects. A memory and a processor as well as other components can include functionality with regard to the figures. The disclosed aspects in connection with the metric devices and/or the system can employ various AI-based schemes for carrying out various aspects thereof. For example, a process for detecting one or more trigger events, reducing a weight of the exercise equipment as a result of the one or more trigger events, modifying one or more reported metrics, and so forth, can be facilitated with an example automatic classifier system and process. In another example, a process for penalizing one weight adjustment while preferring another weight adjustment can be facilitated with the example automatic classifier system and process.

An example classifier can be a function that maps an input attribute vector, x=(x1, x2, x3, x4, xn), to a confidence that the input belongs to a class, that is, f (x)=confidence (class). Such classification can employ a probabilistic and/or statistical-based analysis (e.g., factoring into the analysis repetition and time) to prognose or infer an action that can be automatically performed. In the case of exercise equipment adjustment, for example, attributes can be a weight and a repetition, and the classes can be calories expended and calories required.

A support vector machine (SVM) is an example of a classifier that can be employed. The SVM can operate by finding a hypersurface in the space of possible inputs, which the hypersurface attempts to split the triggering criteria from the non-triggering events. Intuitively, this makes the classification correct for testing data that is near, but not identical to training data. Other directed and undirected model classification approaches include, for example, naïve Bayes, Bayesian networks, decision trees, neural networks, fuzzy logic models, and probabilistic classification models providing different patterns of independence can be employed. Classification as used herein also may be inclusive of statistical regression that is utilized to develop models of priority.

The disclosed aspects can employ classifiers that are explicitly trained (e.g., via generic training data) as well as implicitly trained (e.g., via observing mobile device usage as it relates to triggering events, observing network frequency/technology, receiving extrinsic information, and so on). For example, SVMs can be configured via a learning or training phase within a classifier constructor and feature selection module. Thus, the classifier(s) can be used to automatically learn and perform a number of functions, including but not limited to modifying an intensity associated with exercise equipment, modifying an exercise equipment resistance, and so forth. The criteria can include, but is not limited to, predefined values, attenuation tables or other parameters, service provider preferences and/or policies, and so on.

In one embodiment, described herein is a method comprising generating, by mobile device, a first avatar of a user based on body measurement data, the body measurement data representative of a physique of the user. The method can comprise receiving, by the mobile device, workout data, the workout data representative of a workout that has been performed by the user, wherein the workout is associated with a body part of the user. In response to receiving the workout data, the method can comprise generating, by the mobile device, a second avatar, representative of the user, wherein the second avatar depicts the body part associated with the workout. Additionally, based upon the workout data, the method can comprise modifying, by the mobile device, the body part of the second avatar.

According to another embodiment, a system can facilitate generating a first avatar of a user based on body measurement data, the body measurement data representative of the user. The system can comprise receiving workout data, the workout data representative of a number of weightlifting repetitions that have been performed by the user, wherein the workout is associated with a body part of the user. In response to receiving the workout data, the system can also comprise generating a second avatar, representative of the user, wherein the second avatar depicts the body part associated with the workout. Furthermore, based upon the workout data, the system can comprise altering the body part of the second avatar.

According to yet another embodiment, described herein is a machine-readable storage medium that can perform the operations comprising facilitating generating a first avatar of a user based on body measurement data, the body measurement data representative of a physique of the user. The machine-readable storage medium can perform the operations comprising facilitating receiving workout data, the workout data representative of a workout that has been performed by the user, wherein the workout is associated with a body part of the user. In response to facilitating the receiving of the workout data, the machine-readable storage medium can perform the operations comprising facilitating generating a second avatar, representative of the user, wherein the second avatar depicts the body part associated with the workout. The machine-readable storage medium can perform the operations comprising facilitating modifying the body part of the second avatar based upon the workout data.

These and other embodiments or implementations are described in more detail below with reference to the drawings.

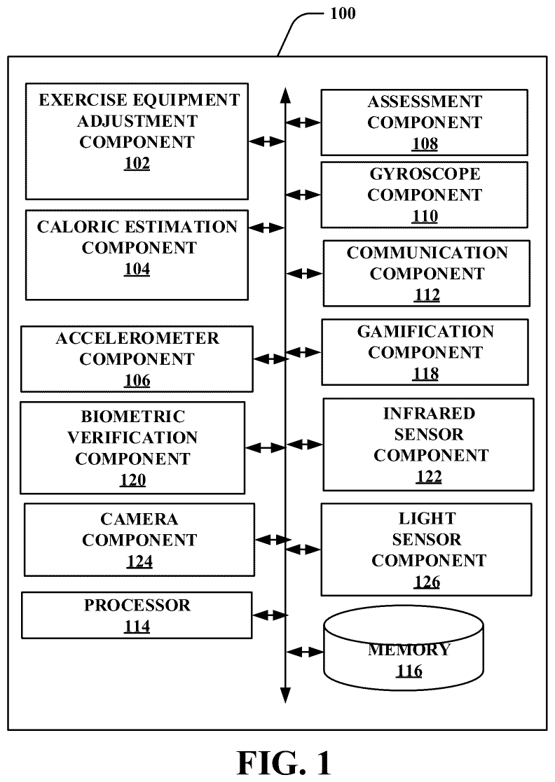

Referring now to FIG. 1, illustrated is an example metric device according to one or more embodiments. In various embodiments, the metric device 100 can be associated with or included in a data analytics system, a data processing system, a graph analytics system, a graph processing system, a big data system, a social network system, a speech recognition system, an image recognition system, a graphical modeling system, a bioinformatics system, a data compression system, an artificial intelligence system, an authentication system, a syntactic pattern recognition system, a medical system, a health monitoring system, a network system, a computer network system, a communication system, a router system, a server system or the like.

The metric device 100 can comprise several components including an exercise equipment adjustment component 102, a caloric estimation component 104, an accelerometer component 106, an assessment component 108, a gyroscope component 110, a communication component 112, an infrared sensor component 122, a biometric verification component 120, a camera component 124, a light sensor component 126, a gamification component 118, a processor 114, and a memory 116. It should be noted that the metric device 100 can also comprise other various sensors, such as pressure sensors for sensing physical pressure, an altimeter for measuring altitude, a humidity sensor for measuring humidity, or the like. The metric device 100 components can be communicatively coupled to each other, wherein bi-directional communication can occur between the components, the processor, and the memory. It should also be noted that various other embodiments are possible that do not comprise all of the aforementioned components. For example, the metric device 100 can comprise the accelerometer component 106, the communication component 112, the infrared sensor component 122, the processor 114, and the memory 116 absent of the other components illustrated in FIG. 1. It should also be noted that in other embodiments, some of the functionality associated with one or more components can be performed at the server device, a virtual reality device, an augmented reality device, a mixed reality device, and/or the mobile device. For example, caloric expenditure data can be generated by the mobile device in response to the mobile device receiving repetition and resistance data from a user workout. Repetitive description of like elements employed in other embodiments described herein is omitted for sake of brevity.

The exercise equipment adjustment component 102 can be configured to adjust a weight and/or resistance associated with the exercise equipment. The weight can be adjusted in response to a mobile device entering a zone associated with the exercise equipment. The weight can also be adjusted in response to a mobile device leaving the zone associated with the exercise equipment.

The exercise equipment adjustment component 102 can generate exercise equipment adjustment data based on size, weight, and/or height (from profile data) of a user. For example, pressure sensors in exercise equipment seats can confirm that the user is seated in a lifting position. A seat, or any adjustable component of the exercise equipment, can be adjusted in response to the metric device's 100 communication with the user's mobile device, wherein the adjustment can be based on the user's profile. In another embodiment, the user can be prompted, via the mobile device, to adjust the seat or any adjustable component of the exercise machine after the mobile device enters the zone of the exercise equipment, the mobile device begins communicating with the metric device 100, and/or there is an indication that the user intends to use the exercise equipment. For example, if the user's profile indicates that the user is 5′11″ in height, then the mobile device can prompt the user (e.g., via audible prompts and/or text on a display screen of the mobile device) to adjust the seat of the exercise equipment to a specific level suitable to the user, based on his/her height. It should be noted that any exercise machine (e.g., preacher curl machine, shoulder lateral raise machine, seated hamstring curl, etc.) with adjustable seats or components (e.g., lock in bar, shoulder press, etc.) can be adjusted based on the aforementioned embodiment. As noted above, the user prompts can be displayed on the user's mobile device and/or audibly communicated to the user via the mobile device. It should be noted that the audible instructions can also be communicated via a wireless or wired headset communicating with the mobile device in this embodiment or any other embodiment. However, in additional embodiments, the instructions can also be displayed on the exercise equipment via a display screen of the exercise equipment.

The caloric estimation component 104 can be configured to approximate a caloric expenditure of a user of the metric devices 100. For instance, calories expended can be based on several factors including, but not limited to: the user's weight, the user's age, the user's body type, the user's height, the resistance of the machine, the repetitions performed by the user at a certain resistance, etc. The metric device 100 can also comprise an accelerometer component 106 for measuring an acceleration associated with use of the exercise equipment. This can provide output data representing how quickly a user performs a set of motions (e.g., up and down motions during a bench press exercise).

An assessment component 108 can assess the metric device 100 and exercise equipment usage and generate data accordingly. For instance, the assessment component 108 can assess the metric device 100 as in use, resting, in communication with another device, etc. The assessment component 108 can also continually assess a user's progress by generating real-time or near real-time data for users during a workout session. The assessment component 108 can comprise sensors (e.g., thermal sensors, heart-rate sensors, pulse sensors, etc.) for detecting physical characteristics of a user. Data generated from the sensors can then be wirelessly communicated to the user's mobile device and/or a server device via a wireless network. Additionally, the assessment component 108 can assess a user's physicality to determine a specific workout plan. For example, based on a user's last workout on a specific exercise machine, the assessment component 108 can provide suggestions to the user based on predictive analysis. If a user can regularly bench two hundred pounds ten times and does not perform a bench press for several months, then the assessment component 108 can predict muscle atrophy based on the user's lapsed time in performing a bench press. For instance, in accordance with other user data inputs (e.g., weight, height, physique, etc.), the assessment component 108 can predict a ten percent muscle atrophy in the user's pectoral muscles based on a two-month lapse in bench press exercises. Conversely, the user profile can allow the user to add additional data that can offset the assessment component's 108 prediction of muscle atrophy. For example, if the user was performing push-ups regularly throughout the two-month bench press lapse, then this could offset the predicted ten percent muscle atrophy by seven percent to only a three percent muscle atrophy.

The metric device 100 can be used to determine health and physicality specific to users. Therefore, a method to securely identify the user that is using the system can be leveraged. For example, biometric data can be used to determine the identity of the user that is actually using the metric device 100 in conjunction with the user's mobile device 206. Biometric security verifications including, but not limited to fingerprint analysis, voice recognition, security codes, facial recognition, eye scan, etc. can be used to confirm the correct user is using the system. The biometric security verifications can be performed at the mobile device and/or via the biometric verification component 120 of the metric device 100. The mobile device and the metric device 100 can communicate such information to each other to allow for confirmation. In certain embodiments, there can be a 2-step verification process (e.g., one verification process at the mobile device 206, and a second verification process at the metric device 100), whereby both verification processes must be complete to confirm the correct user is operating the system.

The metric device 100 can also comprise a gyroscope component 110 for measuring orientation of the metric device 100. Measuring orientation of the metric device 100 can generate exercise data associated with use of the exercise equipment. For example, the gyroscope component 110 can be used to determine the orientation of the exercise equipment relative to the exercise being performed, thereby determining whether the exercise equipment is in use, at rest, extended, protracted, retracted, contracted, up, down, etc. The gyroscope component 110 can also be used to determine the maximum and minimum ranges of motion of a user performing a particular exercise. Additionally, the gyroscope component 110 can be used to determine a user's ability to keep the exercise equipment stable. For example, based on the gyroscope component's 110 orientation during a bench press, user stability data can be generated that can indicate that the user's left arm is stronger than his right arm. Consequently, the user can reduce the bench press weight to accommodate his right arm and/or use this information to begin strengthening his right arm by isolating his right arm during other exercise sessions. The system can also suggest specific exercises for the user to isolate his right arm. This type of localized data regarding the user's strengths and weaknesses can prevent the user from straining or injuring himself. The communication component 112 can be configured to facilitate communication with mobile devices, server devices, other metric devices, wireless network devices, telecommunications network devices, etc. The communication component 112 can comprise a transceiver and a receiver. The communication component 112 can use Wi-Fi, ZigBee, Bluetooth, iBeacon (or similar technology) etc. to facilitate communication with the aforementioned devices. A memory 116 and a processor 114 can comprise functionality with regard to the metric devices 100. Additionally, should communication between a mobile device and the metric devices 100 be interrupted (e.g., by a dropped cellular signal, mobile device battery death, etc.), the memory 116 can store any shared or generated data and/or send the data to a server device. Additionally, the next time that the mobile device is within range of the metric device, the metric device 100 can transmit any previously stored data to the mobile device via the communication component 112. The gamification component 118 can gamify the exercise metrics to generate exercise metric-based challenges to facilitate competition between individuals and/or a group of individuals. Additionally, the infrared sensor component 122 can be configured to measure a distance of the metric device 100 from a location, the user, the mobile device, etc. The infrared sensor component 122 can also be used to determine the amount of weight a user is lifting as discussed later in this disclosure. The camera component 124 can be configured to take photographs and/or video from the metric device 100, and the light sensor component 126 can be configured to interact with a light source associated with the metric device 100.

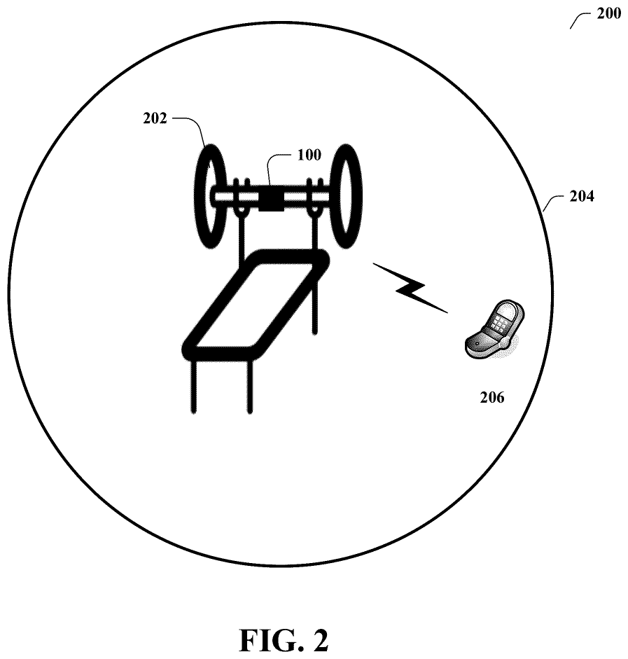

Referring now to FIG. 2, illustrated is an example exercise equipment comprising a metric device 100 in communication with a mobile device 206 according to one or more embodiments. Repetitive description of like elements employed in other embodiments described herein is omitted for sake of brevity. The system 200 can comprise a free weight barbell 202. It should be noted that the metric device 100 can be an add-on component to the free weight barbell 202 or the metric device 100 can be built into the free weight barbell 202. In either scenario, the metric device 100 can be associated with a zone 204 of communication. The zone 204 of communication can establish a distance of communication between the metric device 100 and a mobile device 206 (e.g., via the communication component 112). It should be noted that the center of the radius or the zone 204 can be the metric device 100 itself, or the center can be offset to align with the center of the exercise equipment, thereby generating an equidistant zone around the exercise equipment even if the metric device 100 is attached to a non-center location of the exercise equipment. It should be noted that the zone 204 does not have to be circular but could be any shape or configuration (rectangular, triangular, free form, etc.) Essentially, the mobile device 206 can communicate with the metric devices within zone 204, but the mobile device 206 may not be able to communicate with the metric devices outside of the zone 204 (e.g., via the communication component 112).

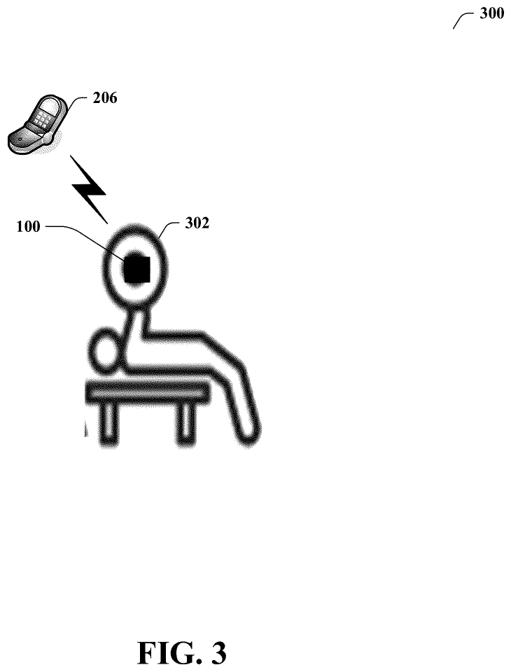

Referring now to FIG. 3, illustrated is an example exercise equipment comprising a metric device in communication with a mobile device according to one or more embodiments. Repetitive description of like elements employed in other embodiments described herein is omitted for sake of brevity. A free weight barbell 302 with an integrated metric device 100 can communicate with the mobile device 206 (e.g., via the communication component 112) in system 300. Communication between the mobile device 206 and the free weight barbell 302 can comprise exercise metrics associated with the user's workout performance. For instance, the exercise metrics can comprise weight and/or resistance data associated with the weight of the barbell and weight plates (e.g., via the assessment component 108), repetition data associated with a number of repetitions the user has completed (e.g., via the accelerometer component 106), and/or user caloric expenditure data (e.g., via the caloric estimation component 104) based on the weight and/or resistance data and repetition data.

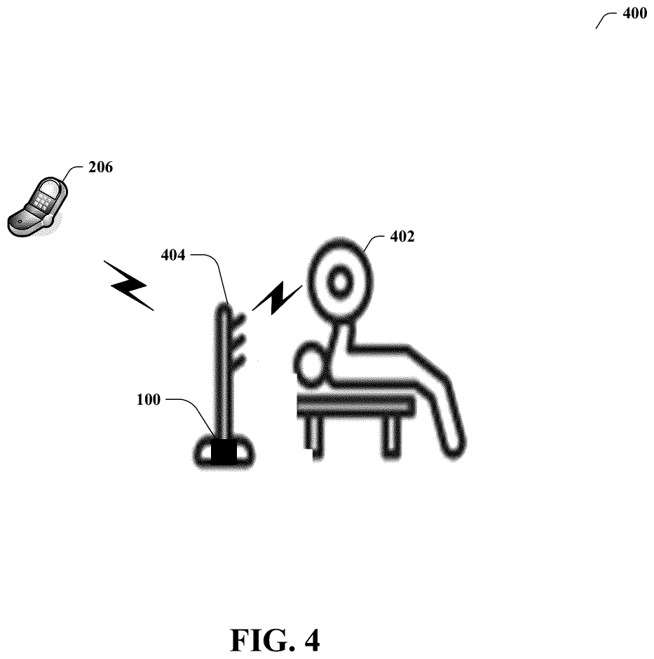

Referring now to FIG. 4, illustrated is an example first exercise equipment in communication with a second exercise equipment comprising a metric device in communication with a mobile device according to one or more embodiments. Repetitive description of like elements employed in other embodiments described herein is omitted for sake of brevity. In another embodiment, a system 400 can comprise multiple exercise devices in communication with each other. For instance, the metric device 100 can be integrated into the weight stand 404 as opposed to the barbell 402. However, the weight stand 404 can communicate with the barbell 402. Therefore, both the weight stand 404 and the barbell 402 can comprise wireless communication devices. For instance, the weight stand 404 and the barbell 402 can both comprise metric devices 100 that can communicate with each other, which can allow the system 400 to determine when the barbell 402 is rested on the weight stand 404 or when the barbell 402 is in use by the user. Consequently, exercise metric data can be sent to the mobile device 206 via the weight stand 404 and barbell 402 metric devices 100.

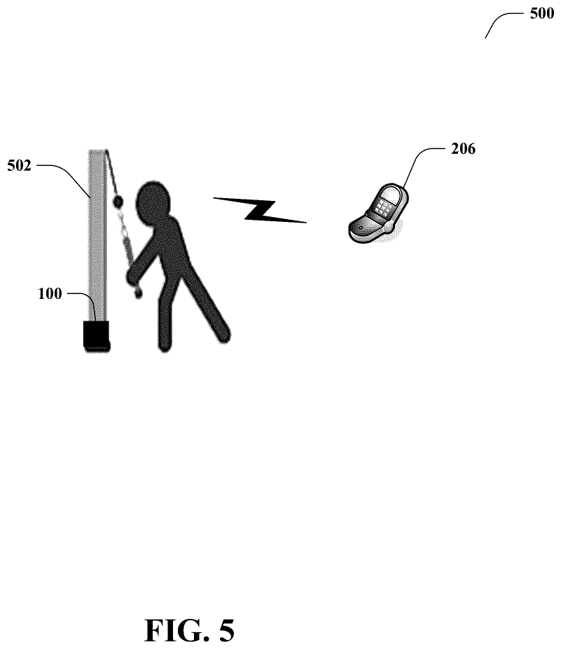

Referring now to FIG. 5, illustrated is an example exercise equipment comprising a metric device in communication with a mobile device according to one or more embodiments. Repetitive description of like elements employed in other embodiments described herein is omitted for sake of brevity. The system 500 can comprise an exercise machine 502 (e.g., cable & pulley, pectoral deck, calf, leg curl, etc.). The exercise machine 502 can comprise the metric device 100, wherein the metric device 100 is internal to the exercise machine 502, wherein the exercise machine 502 was built with (integrated) the metric device 100, and/or wherein the metric device 100 can be externally attached to the exercise machine 502. The metric device 100 can communicate data from a user's workout using the exercise machine 502 with the mobile device 206 of the user.

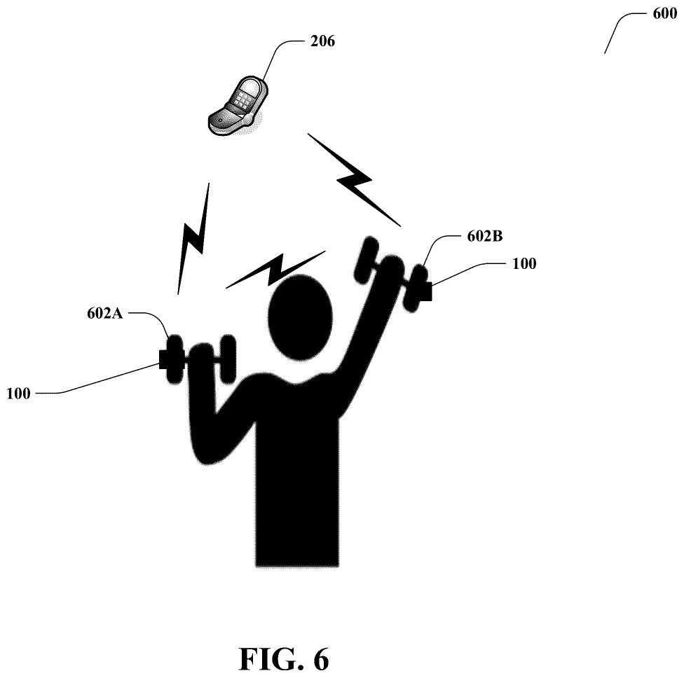

Referring now to FIG. 6, illustrated is an example exercise equipment comprising metric devices in communication with a mobile device according to one or more embodiments. Repetitive description of like elements employed in other embodiments described herein is omitted for sake of brevity. The system 600 can comprise free weights 602A, 602B (e.g., dumbbells, weight plates, etc.). In certain scenarios a user can use multiple exercise equipment simultaneously to facilitate his workout (e.g., using two dumbbells for simultaneous curls). Therefore, both free weights 602A, 602B can comprise metric devices 100 to accurately capture exercise metrics. The metric device 100 associated with free weight 602A can communicate (e.g., via the communication component 112) with another metric device 100 associated with free weight 602B. This can allow the system 600 to infer that the free weights 602A, 602B are being used simultaneously by a single user. Additionally, multiple communication patterns are possible. For instance, the free weight 602A can communicate exercise metrics to the free weight 602B, which can then communicate the 602A exercise metrics and/or the free weight 602B exercise metrics (or vice versa) to the mobile device 206. Alternatively, both free weights 602A, 602B can communicate their respective exercise metrics simultaneously, or near simultaneously, to the mobile device 206. It should also be noted that the communication can be bi-directional (as with any embodiment referenced herein), meaning that the mobile device 206 can also communicate with the free weights 602A, 602B in a similar manner as the free weights 602A, 602B communicate with the mobile device. Bi-directional communication can be used for all other embodiments as well.

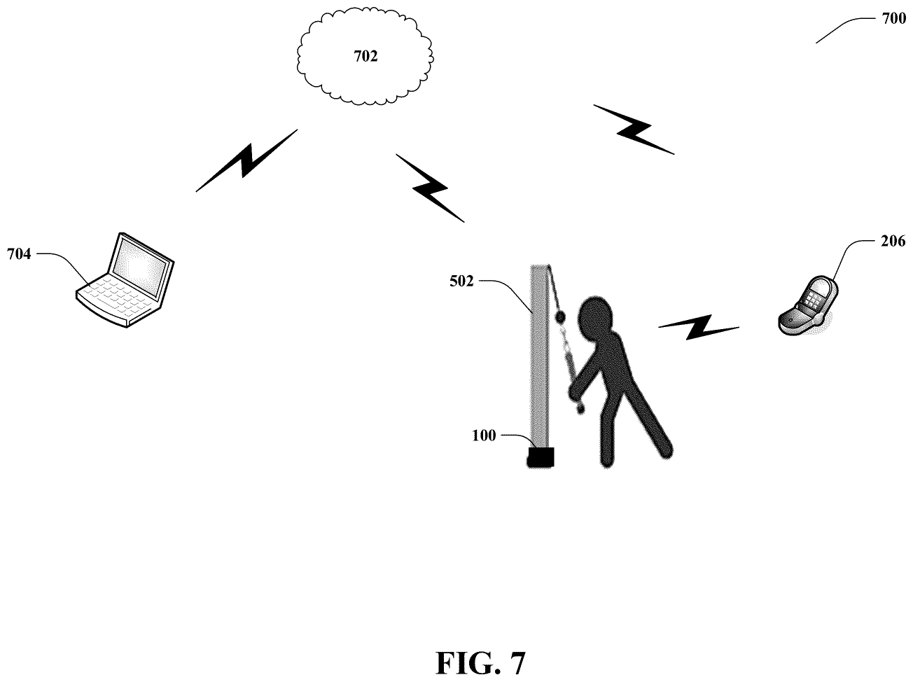

Referring now to FIG. 7, illustrated is an example exercise equipment comprising a metric device in communication with a mobile device and in communication with a wireless network according to one or more embodiments. Repetitive description of like elements employed in other embodiments described herein is omitted for sake of brevity. The system 700 can be employed to use hardware and/or software to solve problems that are highly technical in nature, that are not abstract and that cannot be performed as a set of mental acts by a human. Further, some of the processes performed may be performed by a specialized computer for carrying out defined tasks related to memory operations. The system 700 and/or components of the system can be employed to solve new problems that arise through advancements in technology, computer networks, the Internet and the like.

The system 700 can comprise an exercise machine 502 (e.g., cable & pulley, pectoral deck, calf, leg curl, etc.). It should be noted that exercise machines can include exercise equipment including, but not limited to: cycles, treadmills, pectoral flys, etc. The exercise machine 502 can comprise an internal metric device 100, wherein the exercise machine 502 can be built with the metric device 100, or the metric device 100 can be externally attached to the exercise machine 502. The metric device 100 can communicate with a mobile device 206 of the user.

Additionally, the metric device 100 can communicate exercise metrics to a server or a cloud-based device 702 to facilitate external access to the exercise metrics. For instance, a healthcare provider can monitor a user's exercise history from another mobile device 704, which is remotely located from the exercise machine 502. For purposes of this disclosure, it should be noted that the other mobile device 704 can also be a virtual reality (VR) headset, augmented reality headset (AR), and/or mixed reality (MR) headset. Consequently, the healthcare provider can send exercise regimen data to the exercise machine 502, the metric device 100, and/or the mobile device 206 to prompt the user to alter his or her workout regimen in real-time and/or substantially real-time. The exercise regimen data can also comprise an adjustment of weight and/or resistance of the exercise machine 502. For instance, if the healthcare provider's analysis of the exercise metric data indicates that the user is over-exerting himself in light of a recent surgery, the healthcare provider can remotely adjust a weight and/or resistance of the exercise machine 502 via the other mobile device 704. In like manner, the exercise regimen data can be stored at the server or a cloud-based device 702 for future use or download by the user and/or the exercise machine 502.

In an alternative embodiment, an additional devices can provide data to and receive data from the system 700. For example, a smart watch (not shown) that monitors a user's heart rate can provide heart rate data representative of the user's heart rate to the mobile device 206, 704, the metric device 100, and/or the cloud-based device 702. The heart rate data can then be used to assist the system 700 in generating additional data (e.g., caloric expenditure data).

In another alternative embodiment, a mobile device, VR headset, AR headset, and/or MR headset 704 can provide data to and receive data from the system 700. For example, the system 700 can send workout data, received from the metric device 100 representative of the user's workout, to the mobile device 206. The mobile device 206 can then generate an avatar, based on the user's workout, and send avatar data, representative of the avatar, to the cloud-based device 702, and the cloud-based device 702 can send the avatar data to the VR headset, AR headset, and/or MR headset 704. Likewise, the VR headset, AR headset, and/or MR headset 704 can send data associated with the avatar back to any of the cloud-based device 702, the mobile device 206, and/or the metric device 100.

Thusly, the system 700, can also comprise an avatar generation component that can generate an avatar depicting the physique of the user. The avatar generation component can receive the body measurements of the user via a user profile, such as their height, weight, chest size etc., and accordingly generate an avatar that corresponds to the current body measurements of the user. In some embodiments, the user may input their body measurements via a sliding scale, wherein as the user adjusts the values in the sliding scale, the avatar generation component can adjust the proportions of the avatar in real-time, thereby showcasing to the user how the user's avatar's appearance would change over different body measurement values.

The avatar that is generated upon entering the body measurement values can be referred to as the “baseline avatar.” It should be noted that the avatar itself can be generated or modified at the mobile device 206, 704, server, and/or an MR, AR, VR device in response to receiving data associated with workout data from the metric device 100. As the user proceeds to engage in a workout, workout data is collected from, for example, the metric device 100. Based on this workout data and the type of workout being performed, the avatar generation component can modify the user's baseline avatar to generate a “workout avatar”, wherein the workout avatar depicts the changes to the user's body as a result of the workout. It should also be noted that the baseline avatar can be a default avatar (e.g., previous measurement data is not used to create the baseline avatar) generated by the system and the workout avatar can be a modification of the default avatar. For example, if the user is performing a bicep curl (which primarily targets the biceps), then the assessment component 108 can calculate the number of repetitions being performed, wherein the repetitions further result in an enhancement to the actual size of the user's biceps. Accordingly, in response to receiving such data, the avatar generation component can accentuate the biceps of the workout avatar to illustrate the increase in the size of the user's biceps as a result of the number of repetitions of the bicep curl. The accentuation of the particular body part can be a function of time, number of repetitions, pounds lifted, fatigue, and/or intensity of the workout. Conversely, if the user does not use the metric device 100 for a defined period of time (e.g., two months), then the workout avatar's muscle size can begin to reduce and appear smaller so as to represent muscle atrophy of the user in real life. For instance, if the assessment component 108 determines that there has been a significant rest period in between the user's workouts, the user's workout avatar can automatically be visually modified to indicate muscular atrophy (e.g., the workout avatar's muscles are reducing in size).

The system 700, can include VR, AR, and/or MR headsets for displaying the avatar to the user. For example, if the user is wearing an AR headset, then the user's avatar may be overlaid with other tangible/real-world objects on the real scene in front of the user. In some embodiments, the workout avatar's capabilities within AR, VR, and/or MR can also be associated with a power level number generated by the assessment component 108 and/or the accentuated muscles of the avatar. Upon using the metric device 100, the power level number can be generated by the assessment component 108 based on factors including, but not limited to, resistance, number of repetitions performed within a specific time, fatigue, range of motion, tempo, etc. For example, a higher power level number (e.g., 1200 as opposed to 800) can indicate the strength of an individual as it relates to a specific muscle group. Thus, the power level number can be used to determine how much the workout avatar's muscles should be accentuated. Additionally, the workout avatar's functionality within AR, VR, and/or MR can be based upon the power level number and/or based upon the muscle size of the workout avatar. For example, within an AR, VR, and/or MR boxing game, a workout avatar with a 1200 power level (as based upon an actual work out of the real-life user) might be able to knock a virtual opponent out immediately due to the representative strength of the workout avatar. Whereas, a workout avatar with only an 800 power level (as based upon the actual work out of the real-life user) would not be able to knock a virtual opponent out immediately because they have not yet acquired enough strength as indicated by the data produced from the metric device 100.

In accordance with the embodiments disclosed herein, a method performed by the avatar generation component can be as follows. The method comprises generating a first avatar (e.g., a baseline avatar) based on body measurement data (e.g., height, weight etc.), wherein the body measurement data is representative of a physique of the user. The body measurement data can be received directly from the user, or in some embodiments, based on a body scan of the user. The method also comprises receiving a first set of workout data (e.g., resistance, number of repetitions etc.), wherein the first set of workout data is representative of a workout being performed by the user. The method can comprises associating the workout with at least one body part of the user, which can involve (i) determining the body part(s) that is (are) targeted by said workout and (ii) the number of body parts that are being targeted. For example, in the case of a bicep curl, for point (i), it can be determined that the bicep curl targets the biceps in the arm(s) of the user. Similarly, for point (ii), a bicep curl exercise can involve a user utilizing only a single dumbbell, in which case only the biceps on the arm (i.e., a single arm) lifting the dumbbell would be associated with the bicep curl. The method can also comprise generating a second avatar of the user, wherein the second avatar depicts the at least one body part associated with the workout. Continuing with the previous example of a bicep curl with a single dumbbell, the second avatar would depict the arm, with the biceps of the user, that is lifting the dumbbell. The second avatar can be generated as the user is engaging in the workout. Based on the first set of workout data, the method can comprise accentuating the at least one body part, in the second avatar. The method can also comprise repeating generating of the second avatar of the user and said accentuating the depiction of the at least one body part, until a stop criterion, for detecting an end of the workout, is met. In other words, the method can involve continuing to modify, in real-time, the second avatar as more workout data is collected. However, the modification of the second avatar is to discontinue once a stop criterion is met. In an example embodiment, the stop criterion can be met when it is determined that the user has ceased their workout. The stop criterion can be met when a certain threshold of reps and/or exercises are performed. The stop criterion can remain in place until a new workout has been initiated and/or a rest time duration has been satisfied. The method can comprise displaying the second avatar of the user on a screen of a display device, wherein the display device can include, but is not limited to, at least one of a computer, a mobile device, a VR, an AR, and/or an MR headset. The method can also comprise de-accentuating (i.e., deemphasizing) the at least one body part of the user, in at least one of the first avatar and the second avatar, based upon a determination that a time difference between receiving the first set of workout data and an absence of a second set of workout data, exceeds a threshold. Thus, representing that a significant period of time has lapsed between the user's workouts overall and/or associated with a specific body part. The method can comprise generating a score based on the first set of workout data, wherein the score is indicative of a strength of the second avatar of the user and can also be utilized to determine an accentuation magnitude or a de-accentuation magnitude of the body part of the first or second avatar. The score can be, for example, the power level described earlier herein.

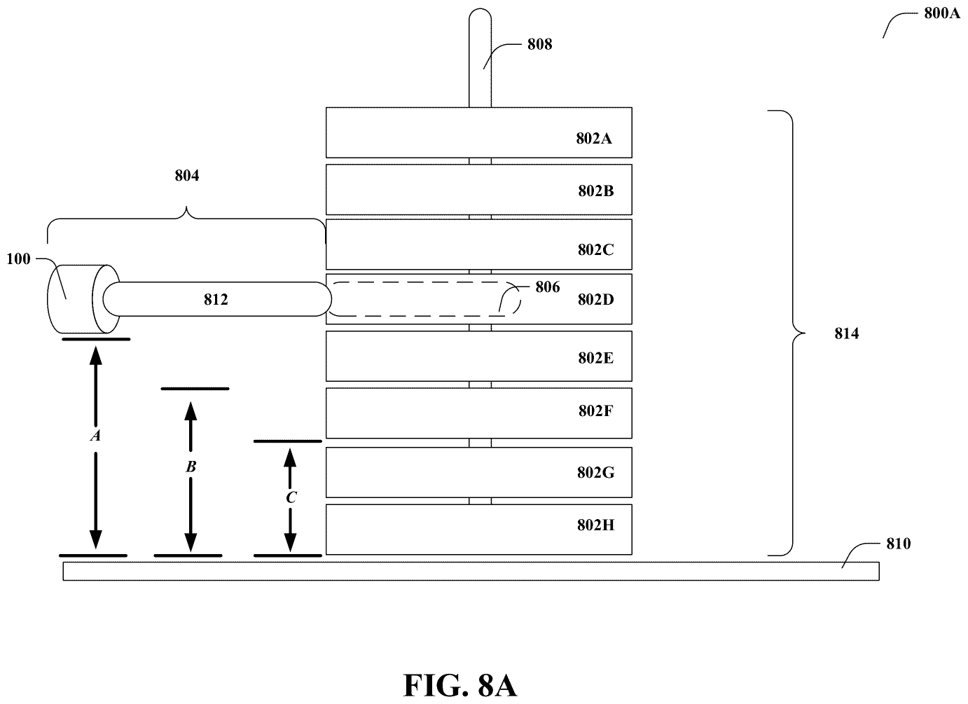

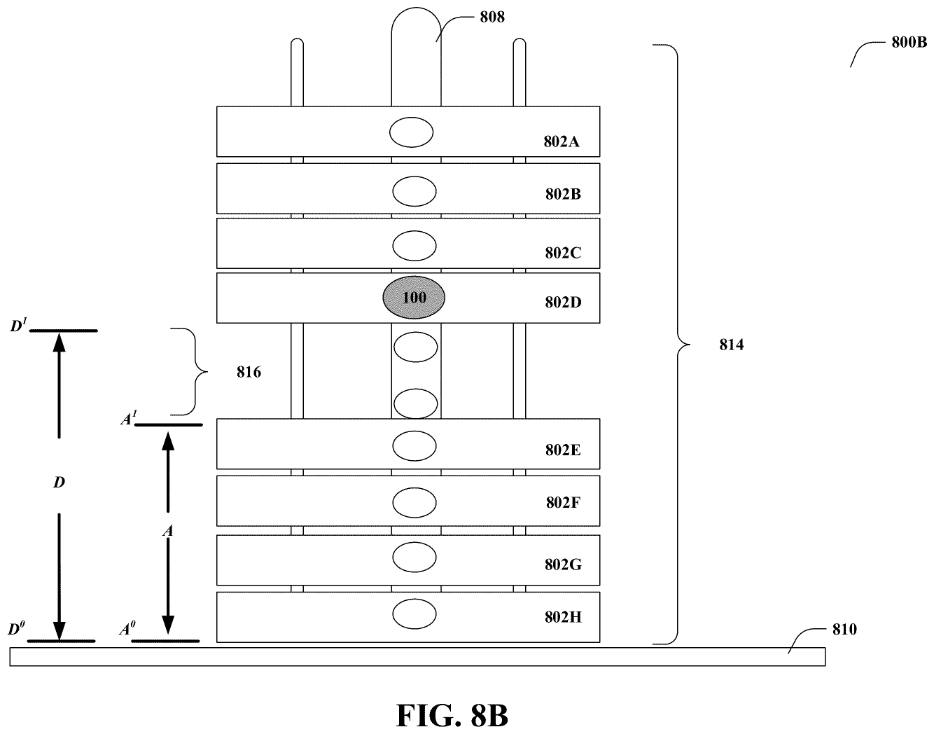

Referring now to FIG. 8A, illustrated is a side view of an example weight stack and metric device according to one or more embodiments. Repetitive description of like elements employed in other embodiments described herein is omitted for sake of brevity.

Automatic identification and data capture (AIDC) refer to the methods of automatically identifying objects, collecting data about them, and entering them directly into computer systems, without human involvement. Technologies typically considered as part of AIDC include, but are not limited to: bar codes, radio frequency identification (RFID), biometrics (e.g., iris and facial recognition systems), magnetic stripes, optical character recognition (OCR), smart cards, and voice recognition. AIDC can be used for determining how much weight a user is attempting to lift and/or how much weight a user is lifting on a weightlifting machine. For example, the weights 802A-H of a weight stack 814 of the exercise machine 502 can comprise one or more identifiers (e.g., color code, RFID tag, location, etc.) and a weight selector pin 804 can comprise the metric device 100 and a pin portion 812. The pin portion and/or the metric device 100 can comprise a magnet for facilitating attaching the weight selector pin 804 to the weight 802A-H. The metric device 100 can also comprise a protective housing to protect the sub-components (e.g., infrared sensor component 122, accelerometer component 106, gyroscope component 110, etc.). The metric device 100 can comprise one or more sensor components (e.g., RFID reader, light sensor, infrared sensor, etc.) that communicate with and/or identify the weights 802A, 802B, 802C, 802D, 802E, 802F, 802G, 802H by the identifiers. Identifiers can be associated with the weights 802A-802H and the identifiers can be communicated within the system 800A. Therefore, the weights 802A-802H that a user is lifting can be determined based on a reference point.

For example, the infrared sensor component 122 can be configured to measure distance. Using a reference point 810 (e.g., floor, base of the exercise machine, etc.), the weights 802A-802H can be various distances from the reference point 810. For example, the weight 802D can be distance A from the reference point 810, the weight 802E can be distance B from the reference point 810, and the weight 802F can be distance C from the reference point 810. Therefore, when the weight selector pin 804 is inserted into a slot 806 of the weight 802D, the distance A can represent the distance of the metric device 100 from the reference point 810, which can be used to determine that the weight 802D has been selected.

Referring now to FIG. 8B, illustrated is a front view of an example weight stack and metric device according to one or more embodiments. Repetitive description of like elements employed in other embodiments described herein is omitted for sake of brevity.

The weight selector pin 804 can comprise various sensor components (e.g., gyroscope component 110, accelerometer component 106, altimeter, infrared sensor component 124, light, etc.) configured to determine how fast the weights 802A-802H are being lifted, how many times the weights 802A-H were lifted, a time duration associated with the weights 802A-802H being lifted or not being lifted, and/or the amount of weight 802A-802H being lifted, etc. Based on a distance from the reference point 810, the infrared sensor component 122 can be used to determine the range of motion of a user while using the exercise machine 502. For example, after the weight selector pin 804 has been inserted into weight 802D and/or the shaft 808, the distance A (e.g., A=A¹−A⁰) from the reference point 810 can represent the weight 802D at rest, and the distance D (e.g., D=D¹−D⁰) from the reference point 810 can represent the weight 802D during an extended range of motion 816 (e.g., D¹−A¹) of the user while using the exercise machine 502. Consequently, when the weight 802D returns to the distance A (reference point A¹, or within a defined tolerance therefrom), the transition from A¹ to D¹ back to A¹ can represent one repetition. The conclusion of one set (e.g., multiple repetitions) can be determined by a time associated with the weight 802D being back at rest at distance A¹. Additionally, the intensity of the user's workout can be calculated based on accelerometer data from the accelerometer component 106 in system 800B. For example, the quicker the acceleration of the weight selector pin 804 during repetitions, the greater the intensity of the workout. Therefore, the accelerometer data can be used to determine one or more metabolic equivalents (METs) associated with the user's workout via the exercise machine 502.

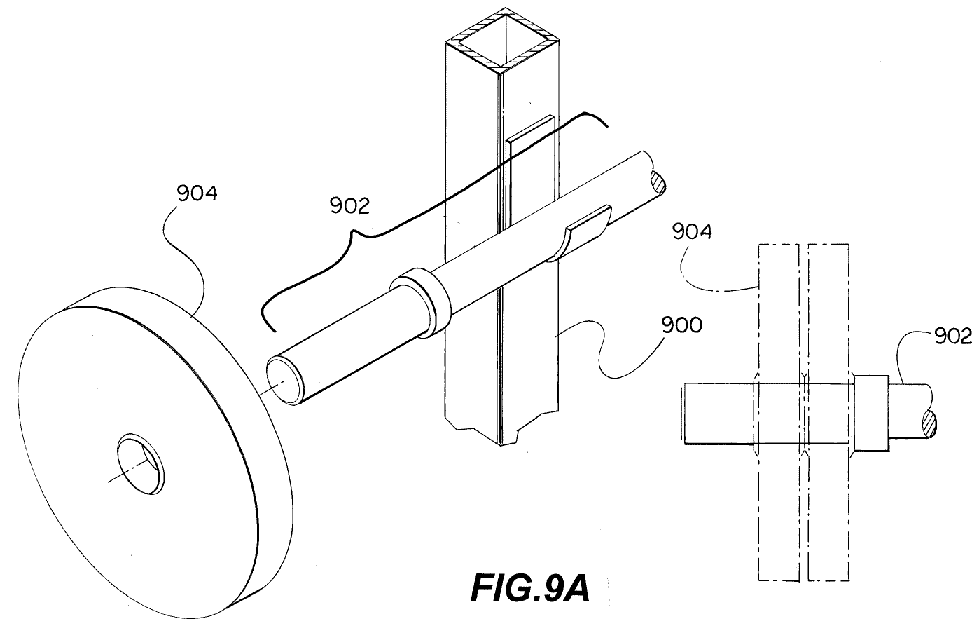

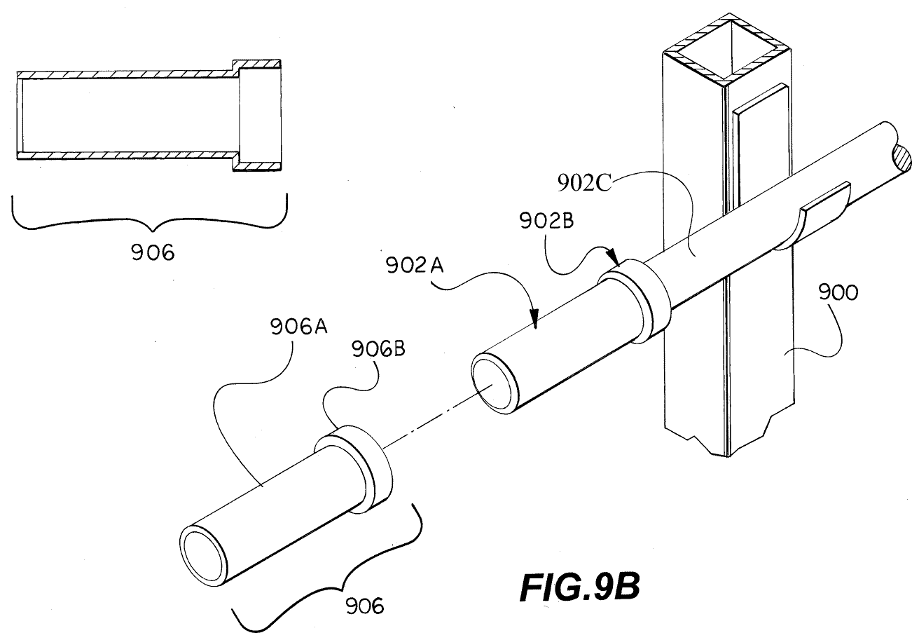

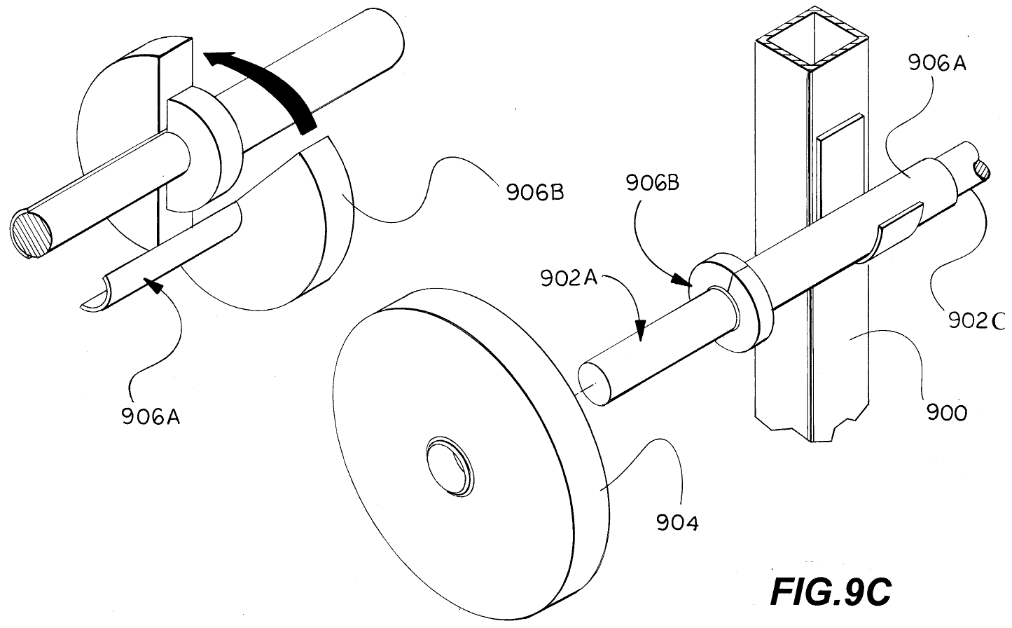

Referring now to FIGS. 9A, 9B, and 9C, illustrated are perspective views of an example barbell, weight plates, weight rack, and a barbell sleeve device (e.g., metric device) according to one or more embodiments. Repetitive description of like elements employed in other embodiments described herein is omitted for sake of brevity.

With regards to FIG. 9A, a weight rack 900 can hold a barbell 902. Weight plates 904 can be added or removed from the barbell 902 while on or off the weight rack 900. A barbell sleeve device 906 (depicted in FIG. 9B) can be adapted to fit or mate with the barbell 902. For example, the barbell 902 can comprise a sleeve 902A, a collar 902B, and a shaft 902C. The barbell sleeve device 906 can also comprise a sleeve 906A and a collar 906B, which can mate with the barbell sleeve 902A and barbell collar 902B, respectively. It should be noted that the sleeve 906A of the barbell sleeve device 906 can also mate with the shaft 902C of the barbell 902 as depicted in FIG. 9B. Additionally, it should be noted that the collar 906B, of the barbell sleeve device 906, can comprise any of the sensors (e.g., altimeter, gyroscope, accelerometer, infrared, etc.) that have been previously discussed with regards to the metric device 100. For instance, the barbell sleeve device 906 can be the metric device 100, adapted to conform to the barbell 902, and comprising pressure sensors, within the sleeve 906A, that are communicatively coupled to the other sensors housed within the collar 906B.

The sleeve 906A, of the barbell sleeve device 906 (e.g., metric device 100), can comprise pressure sensors (e.g., strain gage, scale, capacitors, or the like.). The sleeve 906A of the barbell sleeve device 906 can be so dimensioned to fit around the sleeve 902A of the barbell 902 and/or the shaft 902C of the barbell 902. Once the sleeve 906A of the barbell sleeve device 906 is positioned on the sleeve 902A and/or the shaft 902C of the barbell 902, the pressure sensors can detect the pressure of weight plates 904 applied to the sleeve 906A (illustrated in FIG. 9B) and/or the force of the weight plates as the barbell shaft 902C is forced down in a downward motion against the holding posts on the rack 900 (illustrated in FIG. 9C). It should be noted that the barbell sleeve device 906 can comprise a removable and/or a rechargeable battery system. It should also be noted that the sleeve 906A of the barbell sleeve device 906 can extend the length of the shaft 902C of the barbell 902 such that a user's hands and/or neck can generate pressure on the sleeve 906A during use (e.g., bench press, squats, etc.). In some scenarios, two or more barbell sleeve devices 906 can be used on one barbell 902 to generate data from both sides of the barbell 902.

This pressure sensed by the sleeve 906A of the barbell sleeve device 906 can yield a weight (e.g., pounds, kilograms, etc.) representative of the weight of the weight plates 904 added to the weight of the barbell 902. To determine the weight of the weight plates 904 added, the weight of the barbell 902 can be subtracted from the overall weight perceived by the pressure sensor data. This data can be transmitted from the barbell sleeve device 906 to a user's mobile device 206. In some scenarios, constant use of weight plates 904 can result in inaccurate information. For example, even though a weight plate might say 45 pounds, after years of use, the actual weight plate might only weigh 43 pounds. However, weight thresholds can be predefined to account for the weight loss. For example, a weight threshold might say that any pressure sensed from the barbell sleeve device 906 that would ordinarily register as the 45 pound plate being added can be reduced such that any pressure that is sensed between 43 pounds and 46 pounds would register as the 45 pound weight. In other embodiments, the actual weight of 43 pounds can be transmitted to the mobile device 206 such that the user knows the exact amount of weight he/she is attempting to lift. In certain scenarios, such as with body builders, it can be useful to know that the weight plate that the user believes is providing 45 pounds of resistance, is actually only providing 43 pounds of resistance. The current embodiment can also be used to determine the weight of the weight plates 904 on the barbell 902 during use without a rack 900 (e.g., during a deadlift) based on pressure sensed by the sleeve 906A of the barbell sleeve device 906 when the user picks the barbell 902 up from the ground. When the weight of weight plates 904 applied to the barbell 902 is known prior to the exercise beginning, audible or text alerts can be given to the user to prevent injury. For instance, if the user has only bench pressed 100 pounds based on his/her profile data from previous use of the barbell sleeve device 906, if the pressure sensors of the barbell sleeve device 906 indicate that the user has applied 400 pounds of weight to the barbell 902 he/she intends to use, an alert can be presented to the user to warn of the risk associated therewith.

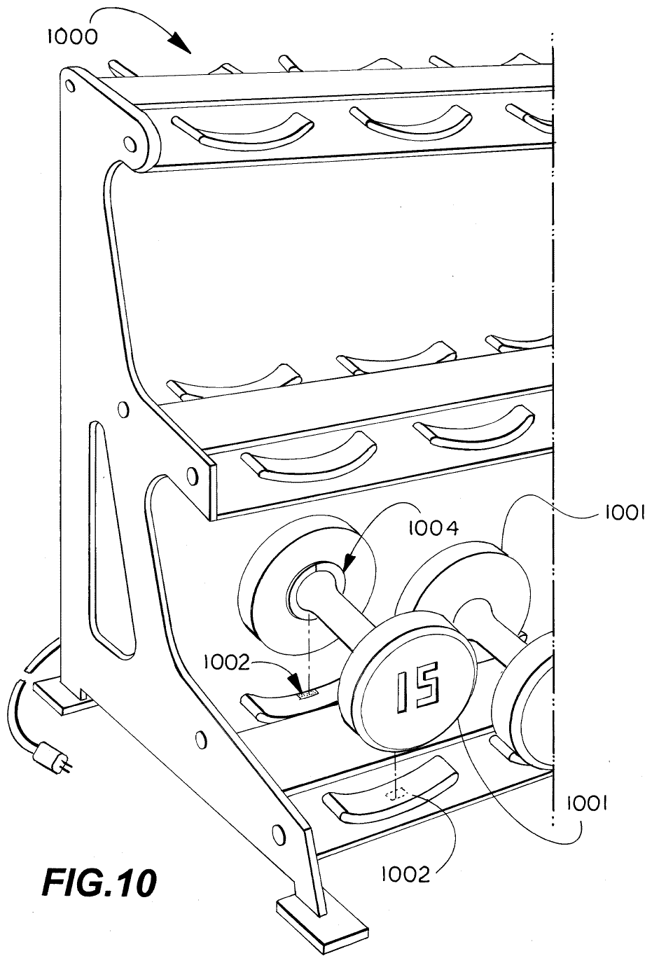

Referring now to FIG. 10, illustrated is a perspective view of an example dumbbell rack comprising dumbbells according to one or more embodiments. Repetitive description of like elements employed in other embodiments described herein is omitted for sake of brevity.

In yet another embodiment, the metric device 100 can take the form of a disk 1004 adapted to fit on a dumbbell 1001. It should be noted that the metric device 100 can be placed at any location on the dumbbell 1001. The dumbbell rack 1000 can be so dimensioned to facilitate wireless charging of the disk 1004 (e.g., metric device 100) while the dumbbells are racked during nonuse. For example, inductors 1002 can be integrated into the dumbbell rack 1000 such that the inductors are touching (or are in close enough proximity to) the disk 1004 to charge the disk 1004 while the dumbbell 1001 is racked. The inductors 1002 can sit in a reservoir capable of receiving a portion of the disk 1004 when the dumbbell 1001 is racked. The dumbbell rack 1000 itself can comprise an electrical cord such that the dumbbell rack 100 can be charged via an electrical outlet, which can provide the electricity needed to charge the disks 1004 through inductance or any other type of wireless charging technology.

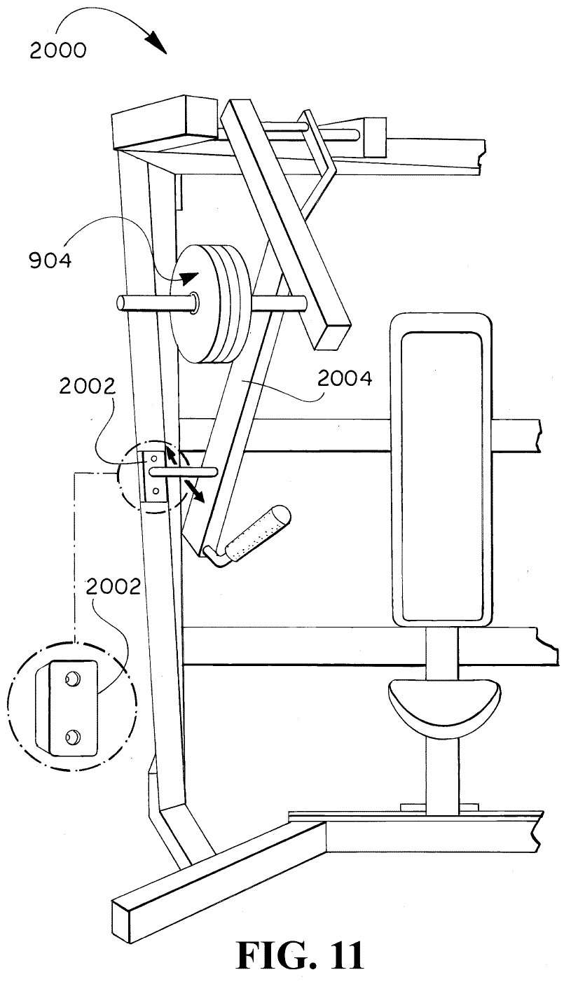

Referring now to FIG. 11, illustrated is a perspective view of an example plate loaded machine according to one or more embodiments. Repetitive description of like elements employed in other embodiments described herein is omitted for sake of brevity.

The plate loaded machine 2000 can comprise an arm 2004 and a bumper device 2002 (e.g., metric device 100). The bumper device 2002 can be so dimensioned as to take on the forms of current bumpers found on plate load machines. The bumper device 2002 can comprise the sensors of the metric device 100. For plate loaded machines, the bumper device 2002 can be and/or comprise a rubber housing (e.g., bumper) such that the bumper device 2002 can support the components of plate loaded machine 2000 while the plate loaded machine 2000 is at rest. The bumper device 2002 can serve as the bumper and comprise springs and/or rubber to protect the internal electronics and absorb impact when the plate loaded machine 2000 is being utilized. Consequently, pressure sensors in the bumper device 2002 can detect the pressure against the bumper device 2002, as weight plates 904 are added to or removed from the horn of the plate loaded machine 2000, to determine the amount of weight added to or removed from the horn. It should be noted that any of the aforementioned systems can also take into account the barbell weight, weight of the plate loaded machine components, etc. for analysis purposes. It should also be noted that the metric device 100 can come in several different form factors including, but not limited to: a weight selector pin for selectorized exercise equipment, a bumper for plate loaded machines, a barbell sleeve device for barbells, and/or a disk for dumbbells.

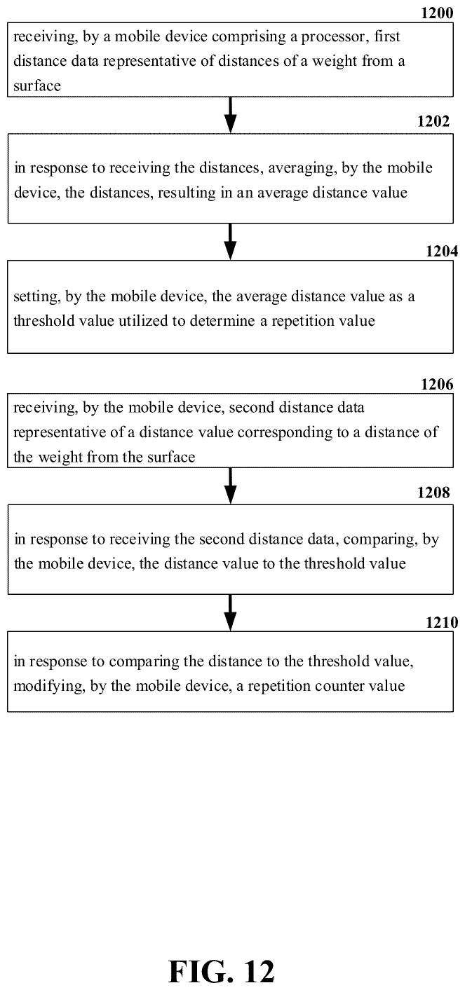

Referring now to FIG. 12, illustrated is an example flow diagram of a method for determining repetition thresholds according to one or more embodiments.

At element 1200, the method can comprise receiving, by a mobile device comprising a processor, first distance data representative of distances of a weight from a surface. In response to receiving the distances, at element 1202, the method can comprise averaging, by the mobile device, the distances, resulting in an average distance value. At element 1204, the method can comprise setting, by the mobile device, the average distance value as a threshold value utilized to determine a repetition value. At element 1206, the method can comprise receiving, by the mobile device, second distance data representative of a distance value corresponding to a distance of the weight from the surface. Additionally, at element 1208, in response to receiving the second distance data, the method can comprise comparing, by the mobile device, the distance value to the threshold value. Furthermore, at element 1210, in response to comparing the distance to the threshold value, the method can comprise modifying, by the mobile device, a repetition counter value.

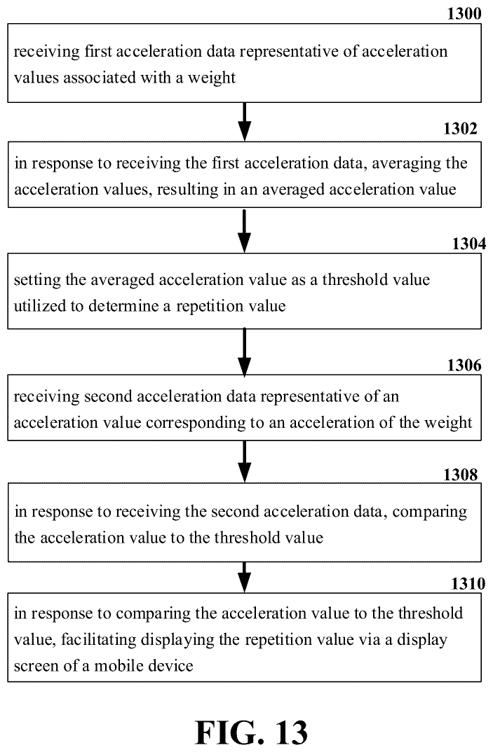

Referring now to FIG. 13, illustrated is an example flow diagram of a system for determining repetition thresholds according to one or more embodiments.

At element 1300, the system can facilitate receiving, first acceleration data representative of acceleration values associated with a weight. In response to receiving the first acceleration data, at element 1302, the system can comprise averaging the acceleration values, resulting in an averaged acceleration value. At element 1304, the system can comprise setting the averaged acceleration value as a threshold value utilized to determine a repetition value. At element 1306, the system can also comprise receiving second acceleration data representative of an acceleration value corresponding to an acceleration of the weight. Furthermore, at element 1308, in response to receiving the second acceleration data, the system can comprise comparing the acceleration value to the threshold value. Furthermore, at element 1310 in response to comparing the acceleration value to the threshold value, the system can comprise facilitating displaying the repetition value via a display screen of a mobile device.

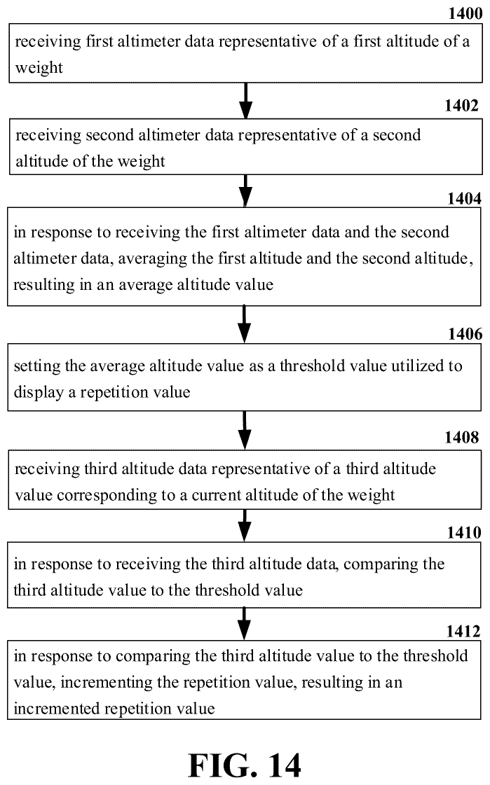

Referring now to FIG. 14, illustrated is an example flow diagram of a machine-readable medium for determining repetition thresholds according to one or more embodiments.

At element 1400, the machine-readable medium can comprise receiving first altimeter data representative of a first altitude of a weight. At element 1402, the machine-readable storage medium can perform the operations comprising receiving second altimeter data representative of a second altitude of the weight. In response to receiving the first altimeter data and the second altimeter data, at element 1404, the machine-readable storage medium can perform the operations comprising averaging the first altitude and the second altitude, resulting in an average altitude value. At element 1406, the machine-readable storage medium can perform the operations comprising setting the average altitude value as a threshold value utilized to display a repetition value. At element 1408, the machine-readable storage medium can perform the operations comprising receiving third altitude data representative of a third altitude value corresponding to a current altitude of the weight. Furthermore, at element 1410, in response to receiving the third altitude data, the machine-readable storage medium can perform the operations comprising comparing the third altitude value to the threshold value. Additionally, at element 1412, in response to comparing the third altitude value to the threshold value, the machine-readable storage medium can perform the operations comprising incrementing the repetition value, resulting in an incremented repetition value.

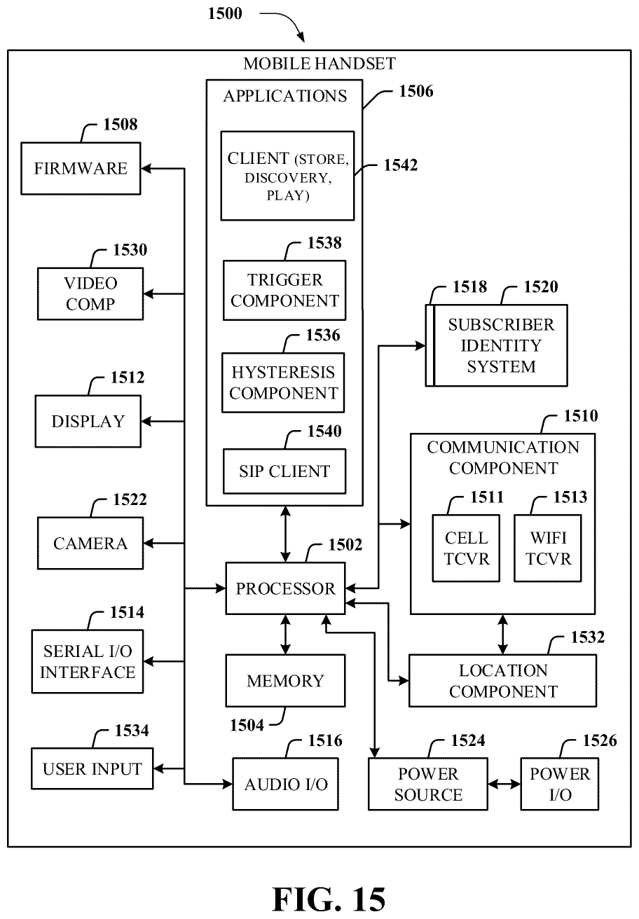

Referring now to FIG. 15, illustrated is a schematic block diagram of an exemplary end-user device such as a mobile device 1500 capable of connecting to a network in accordance with some embodiments described herein. Although a mobile handset 1500 is illustrated herein, it will be understood that other devices can be a mobile device, and that the mobile handset 1500 is merely illustrated to provide context for the embodiments of the various embodiments described herein. The following discussion is intended to provide a brief, general description of an example of a suitable environment in which the various embodiments can be implemented. While the description includes a general context of computer-executable instructions embodied on a machine-readable storage medium, those skilled in the art will recognize that the innovation also can be implemented in combination with other program modules and/or as a combination of hardware and software.

Generally, applications (e.g., program modules) can include routines, programs, components, data structures, etc., that perform particular tasks or implement particular abstract data types. Moreover, those skilled in the art will appreciate that the methods described herein can be practiced with other system configurations, including single-processor or multiprocessor systems, minicomputers, mainframe computers, as well as personal computers, hand-held computing devices, microprocessor-based or programmable consumer electronics, and the like, each of which can be operatively coupled to one or more associated devices.

A computing device can typically include a variety of machine-readable media. Machine-readable media can be any available media that can be accessed by the computer and includes both volatile and non-volatile media, removable and non-removable media. By way of example and not limitation, computer-readable media can comprise computer storage media and communication media. Computer storage media can include volatile and/or non-volatile media, removable and/or non-removable media implemented in any method or technology for storage of information, such as computer-readable instructions, data structures, program modules or other data. Computer storage media can include, but is not limited to, RAM, ROM, EEPROM, flash memory or other memory technology, CD ROM, digital video disk (DVD) or other optical disk storage, magnetic cassettes, magnetic tape, magnetic disk storage or other magnetic storage devices, or any other medium which can be used to store the desired information and which can be accessed by the computer. Aspects of systems, apparatuses, or processes explained in this disclosure can constitute machine-executable component(s) embodied within machine(s), e.g., embodied in one or more computer readable mediums (or media) associated with one or more machines. Such component(s), when executed by the one or more machines, e.g., computer(s), computing device(s), virtual machine(s), etc. can cause the machine(s) to perform the operations described herein.

Communication media typically embodies computer-readable instructions, data structures, program modules or other data in a modulated data signal such as a carrier wave or other transport mechanism, and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

The handset 1500 includes a processor 1502 for controlling and processing all onboard operations and functions. A memory 1504 interfaces to the processor 1502 for storage of data and one or more applications 1506 (e.g., a video player software, user feedback component software, etc.). Other applications can include voice recognition of predetermined voice commands that facilitate initiation of the user feedback signals. The applications 1506 can be stored in the memory 1504 and/or in a firmware 1508, and executed by the processor 1502 from either or both the memory 1504 or/and the firmware 1508. The firmware 1508 can also store startup code for execution in initializing the handset 1500. A communications component 1510 interfaces to the processor 1502 to facilitate wired/wireless communication with external systems, e.g., cellular networks, VoIP networks, and so on. Here, the communications component 1510 can also include a suitable cellular transceiver 1511 (e.g., a GSM transceiver) and/or an unlicensed transceiver 1513 (e.g., Wi-Fi, WiMax) for corresponding signal communications. The handset 1500 can be a device such as a cellular telephone, a PDA with mobile communications capabilities, and messaging-centric devices. The communications component 1510 also facilitates communications reception from terrestrial radio networks (e.g., broadcast), digital satellite radio networks, and Internet-based radio services networks.

The handset 1500 includes a display 1512 for displaying text, images, video, telephony functions (e.g., a Caller ID function), setup functions, and for user input. For example, the display 1512 can also be referred to as a “screen” that can accommodate the presentation of multimedia content (e.g., music metadata, messages, wallpaper, graphics, etc.). The display 1512 can also display videos and can facilitate the generation, editing and sharing of video quotes. A serial I/O interface 1514 is provided in communication with the processor 1502 to facilitate wired and/or wireless serial communications (e.g., USB, and/or IEEE 1394) through a hardwire connection, and other serial input devices (e.g., a keyboard, keypad, and mouse). This supports updating and troubleshooting the handset 1500, for example. Audio capabilities are provided with an audio I/O component 1516, which can include a speaker for the output of audio signals related to, for example, indication that the user pressed the proper key or key combination to initiate the user feedback signal. The audio I/O component 1516 also facilitates the input of audio signals through a microphone to record data and/or telephony voice data, and for inputting voice signals for telephone conversations.

The handset 1500 can include a slot interface 1518 for accommodating a SIC (Subscriber Identity Component) in the form factor of a card Subscriber Identity Module (SIM) or universal SIM 1520, and interfacing the SIM card 1520 with the processor 1502. However, it is to be appreciated that the SIM card 1520 can be manufactured into the handset 1500, and updated by downloading data and software.

The handset 1500 can process IP data traffic through the communication component 1510 to accommodate IP traffic from an IP network such as, for example, the Internet, a corporate intranet, a home network, a person area network, etc., through an ISP or broadband cable provider. Thus, VoIP traffic can be utilized by the handset 1500 and IP-based multimedia content can be received in either an encoded or decoded format.

A video processing component 1522 (e.g., a camera) can be provided for decoding encoded multimedia content. The video processing component 1522 can aid in facilitating the generation, editing and sharing of video quotes. The handset 1500 also includes a power source 1524 in the form of batteries and/or an AC power subsystem, which power source 1524 can interface to an external power system or charging equipment (not shown) by a power I/O component 1526.

The handset 1500 can also include a video component 1530 for processing video content received and, for recording and transmitting video content. For example, the video component 1530 can facilitate the generation, editing and sharing of video quotes. A location tracking component 1532 facilitates geographically locating the handset 1500. As described hereinabove, this can occur when the user initiates the feedback signal automatically or manually. A user input component 1534 facilitates the user initiating the quality feedback signal. The user input component 1534 can also facilitate the generation, editing and sharing of video quotes. The user input component 1534 can include such conventional input device technologies such as a keypad, keyboard, mouse, stylus pen, and/or touch screen, for example.

Referring again to the applications 1506, a hysteresis component 1536 facilitates the analysis and processing of hysteresis data, which is utilized to determine when to associate with the access point. A software trigger component 1538 can be provided that facilitates triggering of the hysteresis component 1538 when the Wi-Fi transceiver 1513 detects the beacon of the access point. A SIP client 1540 enables the handset 1500 to support SIP protocols and register the subscriber with the SIP registrar server. The applications 1506 can also include a client 1542 that provides at least the capability of discovery, play and store of multimedia content, for example, music.

The handset 1500, as indicated above related to the communications component 1510, includes an indoor network radio transceiver 1513 (e.g., Wi-Fi transceiver). This function supports the indoor radio link, such as IEEE 802.11, for the dual-mode GSM handset 1500. The handset 1500 can accommodate at least satellite radio services through a handset that can combine wireless voice and digital radio chipsets into a single handheld device.

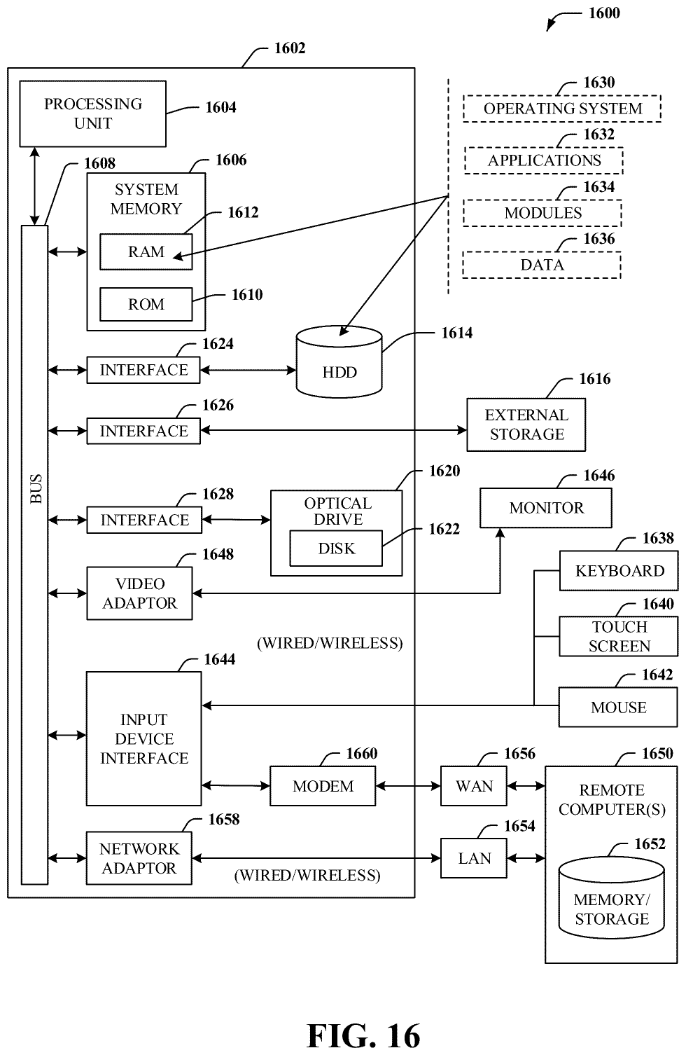

In order to provide additional context for various embodiments described herein, FIG. 16 and the following discussion are intended to provide a brief, general description of a suitable computing environment 1600 in which the various embodiments of the embodiment described herein can be implemented. While the embodiments have been described above in the general context of computer-executable instructions that can run on one or more computers, those skilled in the art will recognize that the embodiments can be also implemented in combination with other program modules and/or as a combination of hardware and software.

Generally, program modules include routines, programs, components, data structures, etc., that perform particular tasks or implement particular abstract data types. Moreover, those skilled in the art will appreciate that the disclosed methods can be practiced with other computer system configurations, including single-processor or multiprocessor computer systems, minicomputers, mainframe computers, Internet of Things (IoT) devices, distributed computing systems, as well as personal computers, hand-held computing devices, microprocessor-based or programmable consumer electronics, and the like, each of which can be operatively coupled to one or more associated devices.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically include a variety of media, which can include computer-readable media, machine-readable media, and/or communications media, which two terms are used herein differently from one another as follows. Computer-readable media or machine-readable media can be any available media that can be accessed by the computer and includes both volatile and nonvolatile media, removable and non-removable media. By way of example, and not limitation, computer-readable media or machine-readable media can be implemented in connection with any method or technology for storage of information such as computer-readable or machine-readable instructions, program modules, structured data or unstructured data.

Computer-readable storage media can include, but are not limited to, random access memory (RAM), read only memory (ROM), electrically erasable programmable read only memory (EEPROM), flash memory or other memory technology, compact disk read only memory (CD-ROM), digital versatile disk (DVD), Blu-ray disc (BD) or other optical disk storage, magnetic cassettes, magnetic tape, magnetic disk storage or other magnetic storage devices, solid state drives or other solid state storage devices, or other tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

With reference again to FIG. 16, the example environment 1600 for implementing various embodiments of the aspects described herein includes a computer 1602, the computer 1602 including a processing unit 1604, a system memory 1606 and a system bus 1608. The system bus 1608 couples system components including, but not limited to, the system memory 1606 to the processing unit 1604. The processing unit 1604 can be any of various commercially available processors. Dual microprocessors and other multi-processor architectures can also be employed as the processing unit 1604.

The system bus 1608 can be any of several types of bus structure that can further interconnect to a memory bus (with or without a memory controller), a peripheral bus, and a local bus using any of a variety of commercially available bus architectures. The system memory 1606 includes ROM 1610 and RAM 1612. A basic input/output system (BIOS) can be stored in a non-volatile memory such as ROM, erasable programmable read only memory (EPROM), EEPROM, which BIOS contains the basic routines that help to transfer information between elements within the computer 1602, such as during startup. The RAM 1612 can also include a high-speed RAM such as static RAM for caching data.

The computer 1602 further includes an internal hard disk drive (HDD) 1614 (e.g., EIDE, SATA), one or more external storage devices 1616 (e.g., a magnetic floppy disk drive (FDD) 1616, a memory stick or flash drive reader, a memory card reader, etc.) and an optical disk drive 1620 (e.g., which can read or write from a CD-ROM disc, a DVD, a BD, etc.). While the internal HDD 1614 is illustrated as located within the computer 1602, the internal HDD 1614 can also be configured for external use in a suitable chassis (not shown). Additionally, while not shown in environment 1600, a solid-state drive (SSD) could be used in addition to, or in place of, an HDD 1614. The HDD 1614, external storage device(s) 1616 and optical disk drive 1620 can be connected to the system bus 1608 by an HDD interface 1624, an external storage interface 1626 and an optical drive interface 1628, respectively. The interface 1624 for external drive implementations can include at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 1602, the drives and storage media accommodate the storage of any data in a suitable digital format. Although the description of computer-readable storage media above refers to respective types of storage devices, it should be appreciated by those skilled in the art that other types of storage media which are readable by a computer, whether presently existing or developed in the future, could also be used in the example operating environment, and further, that any such storage media can contain computer-executable instructions for performing the methods described herein.

A number of program modules can be stored in the drives and RAM 1612, including an operating system 1630, one or more application programs 1632, other program modules 1634 and program data 1636. All or portions of the operating system, applications, modules, and/or data can also be cached in the RAM 1612. The systems and methods described herein can be implemented utilizing various commercially available operating systems or combinations of operating systems.

Computer 1602 can optionally include emulation technologies. For example, a hypervisor (not shown) or other intermediary can emulate a hardware environment for operating system 1630, and the emulated hardware can optionally be different from the hardware illustrated in FIG. 16. In such an embodiment, operating system 1630 can include one virtual machine (VM) of multiple VMs hosted at computer 1602. Furthermore, operating system 1630 can provide runtime environments, such as the Java runtime environment or the .NET framework, for applications 1632. Runtime environments are consistent execution environments that allow applications 1632 to run on any operating system that includes the runtime environment. Similarly, operating system 1630 can support containers, and applications 1632 can be in the form of containers, which are lightweight, standalone, executable packages of software that include, e.g., code, runtime, system tools, system libraries and settings for an application.

Further, computer 1602 can be enable with a security module, such as a trusted processing module (TPM). For instance, with a TPM, boot components hash next in time boot components, and wait for a match of results to secured values, before loading a next boot component. This process can take place at any layer in the code execution stack of computer 1602, e.g., applied at the application execution level or at the operating system (OS) kernel level, thereby enabling security at any level of code execution.

A user can enter commands and information into the computer 1602 through one or more wired/wireless input devices, e.g., a keyboard 1638, a touch screen 1640, and a pointing device, such as a mouse 1642. Other input devices (not shown) can include a microphone, an infrared (IR) remote control, a radio frequency (RF) remote control, or other remote control, a joystick, a virtual reality controller and/or virtual reality headset, a game pad, a stylus pen, an image input device, e.g., camera(s), a gesture sensor input device, a vision movement sensor input device, an emotion or facial detection device, a biometric input device, e.g., fingerprint or iris scanner, or the like. These and other input devices are often connected to the processing unit 1604 through an input device interface 1644 that can be coupled to the system bus 1608, but can be connected by other interfaces, such as a parallel port, an IEEE 1394 serial port, a game port, a USB port, an IR interface, a BLUETOOTH® interface, etc.

A monitor 1646 or other type of display device can be also connected to the system bus 1608 via an interface, such as a video adapter 1648. In addition to the monitor 1646, a computer typically includes other peripheral output devices (not shown), such as speakers, printers, etc.

The computer 1602 can operate in a networked environment using logical connections via wired and/or wireless communications to one or more remote computers, such as a remote computer(s) 1650. The remote computer(s) 1650 can be a workstation, a server computer, a router, a personal computer, portable computer, microprocessor-based entertainment appliance, a peer device or other common network node, and typically includes many or all of the elements described relative to the computer 1602, although, for purposes of brevity, only a memory/storage device 1652 is illustrated. The logical connections depicted include wired/wireless connectivity to a local area network (LAN) 1654 and/or larger networks, e.g., a wide area network (WAN) 1656. Such LAN and WAN networking environments are commonplace in offices and companies, and facilitate enterprise-wide computer networks, such as intranets, all of which can connect to a global communications network, e.g., the Internet.

When used in a LAN networking environment, the computer 1602 can be connected to the local network 1654 through a wired and/or wireless communication network interface or adapter 1658. The adapter 1658 can facilitate wired or wireless communication to the LAN 1654, which can also include a wireless access point (AP) disposed thereon for communicating with the adapter 1658 in a wireless mode.

When used in a WAN networking environment, the computer 1602 can include a modem 1660 or can be connected to a communications server on the WAN 1656 via other means for establishing communications over the WAN 1656, such as by way of the Internet. The modem 1660, which can be internal or external and a wired or wireless device, can be connected to the system bus 1608 via the input device interface 1644. In a networked environment, program modules depicted relative to the computer 1602 or portions thereof, can be stored in the remote memory/storage device 1652. It will be appreciated that the network connections shown are example and other means of establishing a communications link between the computers can be used.

When used in either a LAN or WAN networking environment, the computer 1602 can access cloud storage systems or other network-based storage systems in addition to, or in place of, external storage devices 1616 as described above. Generally, a connection between the computer 1602 and a cloud storage system can be established over a LAN 1654 or WAN 1656 e.g., by the adapter 1658 or modem 1660, respectively. Upon connecting the computer 1602 to an associated cloud storage system, the external storage interface 1626 can, with the aid of the adapter 1658 and/or modem 1660, manage storage provided by the cloud storage system as it would other types of external storage. For instance, the external storage interface 1626 can be configured to provide access to cloud storage sources as if those sources were physically connected to the computer 1602.

The computer 1602 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, store shelf, etc.), and telephone. This can include Wireless Fidelity (Wi-Fi) and BLUETOOTH® wireless technologies. Thus, the communication can be a predefined structure as with a conventional network or simply an ad hoc communication between at least two devices.

The computer is operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This includes at least Wi-Fi and Bluetooth™ wireless technologies. Thus, the communication can be a predefined structure as with a conventional network or simply an ad hoc communication between at least two devices.

Wi-Fi, or Wireless Fidelity, allows connection to the Internet from a couch at home, a bed in a hotel room, or a conference room at work, without wires. Wi-Fi is a wireless technology similar to that used in a cell phone that enables such devices, e.g., computers, to send and receive data indoors and out; anywhere within the range of a base station. Wi-Fi networks use radio technologies called IEEE 802.11 (a, b, g, etc.) to provide secure, reliable, fast wireless connectivity. A Wi-Fi network can be used to connect computers to each other, to the Internet, and to wired networks (which use IEEE 802.3 or Ethernet). Wi-Fi networks operate in the unlicensed 2.4 and 5 GHz radio bands, at an 11 Mbps (802.11a) or 54 Mbps (802.11b) data rate, for example, or with products that contain both bands (dual band), so the networks can provide real-world performance similar to the basic 10 BaseT wired Ethernet networks used in many offices.

The above description of illustrated embodiments of the subject disclosure, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as those skilled in the relevant art can recognize.

In this regard, while the subject matter has been described herein in connection with various embodiments and corresponding FIGs, where applicable, it is to be understood that other similar embodiments can be used or modifications and additions can be made to the described embodiments for performing the same, similar, alternative, or substitute function of the disclosed subject matter without deviating therefrom. Therefore, the disclosed subject matter should not be limited to any single embodiment described herein, but rather should be construed in breadth and scope in accordance with the appended claims below.

What is claimed is:

1. A method, comprising:
generating, by mobile device, a first avatar of a user based on body measurement data, the body measurement data representative of a physique of the user;
receiving, by the mobile device, workout data, the workout data representative of a workout that has been performed by the user, wherein the workout is associated with a body part of the user;
in response to receiving the workout data, generating, by the mobile device, a second avatar, representative of the user, wherein the second avatar depicts the body part associated with the workout; and
based upon the workout data, modifying, by the mobile device, the body part of the second avatar.

2. The method of claim 1, wherein the modifying the body part of the second avatar comprises accentuating the body part of the second avatar.

3. The method of claim 1, wherein the modifying the body part of the second avatar comprises de-accentuating the body part of the second avatar.

4. The method of claim 1, further comprising:
modifying, by the mobile device, the body part of the second avatar until a stop criterion has been satisfied.

5. The method of claim 1, wherein the workout data comprises a power level number.

6. The method of claim 1, further comprising:
in response to the mobile device being determined to be a defined distance from an apparatus, receiving, by the mobile device via a transceiver, a wireless signal from the apparatus,
wherein the apparatus comprises an accelerometer configured to generate acceleration data associated with a motion of the user.

7. The method of claim 1, further comprising:
sending, by the mobile device, the second avatar of the user to be displayed on a screen of a display device, wherein the display device includes at least one of:
a virtual reality (VR) headset;
an augmented reality (AR) headset; or
a mixed reality (MR) headset.

8. The method of claim 1, wherein the workout data is first workout data, further comprising:
based on a time difference between receiving the first workout data and receiving second workout data, de-accentuating, by the mobile device, the body part of the second avatar.

9. The method of claim 1, further comprising:
generating, by the mobile device, a score based on the workout data, wherein the score is indicative of a strength of the second avatar of the user.

10. The method of claim 1, wherein the body part of the user is a bicep of the user.

11. A system, comprising:
a processor; and
a memory that stores executable instructions that, when executed by the processor, facilitate performance of operations, comprising:
generating a first avatar of a user based on body measurement data, the body measurement data representative of the user;
receiving workout data, the workout data representative of a number of weightlifting repetitions of a workout that has been performed by the user, wherein the workout is associated with a body part of the user;
in response to receiving the workout data, generating a second avatar, representative of the user, wherein the second avatar depicts the body part associated with the workout; and
based upon the workout data, altering the body part of the second avatar.

12. The system of claim 11, wherein the altering the body part of the second avatar comprises accentuating the body part of the second avatar.

13. The system of claim 11, wherein the altering the body part of the second avatar ceases after a stop criterion has been satisfied.

14. The system of claim 11, wherein the executable instructions, when executed by the processor, facilitate the performance of the operations, further comprising:
in response a mobile device being determined to be a defined distance from a metric device, receiving, by the system via a transceiver, a wireless signal from the metric device, wherein the metric device comprises an accelerometer configured to generate acceleration data associated with a motion of the user.

15. The system of claim 11, wherein the executable instructions, when executed by the processor, facilitate the performance of operations, further comprising:
sending the second avatar of the user to be displayed on a screen of a display device, wherein the display device includes at least one of:
a virtual reality (VR) headset;
an augmented reality (AR) headset; or
a mixed reality (MR) headset.

16. The system of claim 15, wherein the workout data is first workout data, and based on a time difference between receiving the first workout data and receiving second workout data, the altering the body part of the second avatar comprises de-accentuating the body part of the second avatar.

17. A machine-readable storage medium, comprising executable instructions that, when executed by a processor, facilitate performance of operations, comprising:
facilitating generating a first avatar of a user based on body measurement data, the body measurement data representative of a physique of the user;
facilitating receiving workout data, the workout data representative of a workout that has been performed by the user, wherein the workout is associated with a body part of the user;
in response to facilitating the receiving of the workout data, facilitating generating a second avatar, representative of the user, wherein the second avatar depicts the body part associated with the workout; and
facilitating modifying the body part of the second avatar based upon the workout data.

18. The machine-readable storage medium of claim 17, wherein the modifying the body part of the second avatar comprises facilitating at least one of:
accentuating the body part of the second avatar; or
de-accentuating the body part of the second avatar.

19. The machine-readable storage medium of claim 18, wherein the executable instructions, when executed by the processor, facilitate the performance of operations, further comprising:
facilitating sending the second avatar of the user to be displayed on a screen of a display device, wherein the display device includes at least one of:
a virtual reality (VR) headset;
an augmented reality (AR) headset; or
a mixed reality (MR) headset.

20. The machine-readable storage medium of claim 19, wherein the workout data is first workout data, and wherein the facilitating de-accentuating the body part of the second avatar is based on a time difference between receiving the first workout data and receiving second workout data.
